SEQUENCE LISTING (31 sequences)

SEQ ID NO: 1 - protein, 1273 aa, Artificial Sequence
  Original S glycoprotein (Wuhan strain, MN988669)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 222 aa, Artificial Sequence
  M from Wuhan strain (MN988669)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 75 aa, Artificial Sequence
  E from Wuhan strain (MN988669)
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 419 aa, Artificial Sequence
  N from Wuhan strain (MN988669)
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 1273 aa, Artificial Sequence
  S glycoprotein with Triple mutations and S1-S2 mutation (Wuhan strain, MN988669)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGCEHVNNCYECDIPIGAGICASYQTQTNSPSGSASVASQSIICYTCCLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1270 aa, Artificial Sequence
  S glycoprotein from South Africa strain with mutations (B.1.351)
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPSGSASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1273 aa, Artificial Sequence
  S glycoprotein from India strain with mutations ((B .1. 617)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSGSASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 3822 bp, Artificial Sequence
  Original S glycoprotein (Wuhan strain, MN988669)
atgttcgtgttcctcgtgctgctgcccctggtgtctagccagtgcgtgaacctgaccaccagaacccagctgcctccagcctacaccaacagcttcaccaggggagtgtactaccccgacaaggtgttccggagcagcgtgctgcatagcacccaggacctgttcctgcccttcttcagcaacgtgacttggttccacgccatccacgtcagcggaaccaacggcaccaagcgctttgacaacccagtgctgcccttcaacgacggagtgtacttcgccagcaccgagaagagcaacatcatccggggttggatcttcggcaccacactggacagcaagacccagagcctgctgatcgtgaacaacgccaccaacgtcgtgatcaaggtctgcgagttccagttttgcaacgaccccttcctgggcgtgtactaccacaagaacaacaagtcttggatggagagcgagttccgggtgtacagcagcgccaacaattgcaccttcgagtacgtgtcccagcccttcctgatggacctggaaggcaagcagggcaacttcaagaacctgcgggagttcgtgttcaagaacatcgacggctacttcaagatctacagcaagcacacccccatcaacctcgtgagagacctgccacagggcttcagcgctctggaacctctggtggatctgccaatcggcatcaacatcacccggttccagaccctgctggctctgcacagaagctacctgaccccaggcgattcttctagcggctggacagcaggagccgccgcttattacgtgggctatctgcagccccggaccttcctgctgaagtacaacgagaacggcaccatcaccgacgcagtggattgcgctctggatcctctgagcgagaccaagtgcaccctgaagagcttcaccgtggagaagggcatctaccagaccagcaacttccgggtgcagcctacagagagcatcgtgcggttccctaacatcaccaacctctgccccttcggcgaggtgttcaacgccaccagattcgccagcgtctacgcctggaaccggaagcggatcagcaattgcgtggccgactacagcgtgctgtacaacagcgccagcttcagcaccttcaagtgctacggcgtgtcccctaccaagctgaacgatctgtgcttcaccaacgtgtacgccgacagcttcgtgatcaggggcgacgaagtgcgacagatcgccccaggacagacaggaaagatcgccgattacaactacaagctgcccgacgacttcaccggctgcgtgatcgcttggaacagcaacaacctggacagcaaggtcggcggcaactacaactacctgtaccggctgttccggaagagcaacctgaagcccttcgagcgggacatcagcacagagatctaccaggccggcagcacaccttgtaacggcgtggagggcttcaactgctacttccctctgcagagctacggcttccagcctacaaacggcgtgggataccagccttacagggtggtggtgctgagcttcgaactgctgcacgctccagccaccgtctgcggacctaaaaagagcaccaacctggtgaagaacaagtgcgtgaatttcaacttcaacggcctgaccggaaccggcgtgctgaccgagagcaacaagaagttcctgcccttccagcagttcggcagagacatcgcagacaccacagacgccgtgagagatcctcagaccctggagatcctggacatcaccccttgcagctttggcggagtgtccgtgatcaccccaggaaccaacaccagcaatcaggtggccgtgctgtaccaggacgtgaattgcaccgaggtgccagtggccattcacgcagatcagctgacccctacctggagagtgtacagcaccggaagcaacgtgttccagaccagggcaggttgcctgatcggagccgagcacgtgaacaacagctacgagtgcgacatccccatcggcgccggcatttgcgcctcttaccagacccagaccaacagccctagaagagccagaagcgtggcctctcagagcatcatcgcctacaccatgagcctgggagccgagaacagcgtggcctacagcaacaacagcatcgccatccccaccaacttcaccatcagcgtgaccaccgagattctgcccgtgtctatgaccaagaccagcgtcgactgcaccatgtacatctgcggcgacagcaccgagtgctctaacctgctgctgcagtacggctccttttgcacccagctgaacagggctctgacaggcatcgcagtggagcaggacaagaacacccaggaggtcttcgcccaggtcaagcagatctacaagaccccccctatcaaggacttcggcggctttaacttcagccagatcctgcccgatcccagcaagcctagcaagcggagcttcatcgaggacctgctgttcaacaaggtcaccctggccgacgcaggcttcatcaagcagtacggcgattgcctgggcgacattgccgccagagacctgatttgcgcccagaagttcaacggcctgacagtgctgcctcctctgctgaccgacgagatgatcgcccagtatacaagcgctctgctggcaggaaccatcaccagcggttggacctttggagccggagccgctctgcagatcccttttgccatgcagatggcctaccgcttcaacggaatcggcgtgacccagaacgtgctctacgagaaccagaagctgatcgccaaccagttcaacagcgccatcggcaagatccaggacagcctgagcagcacagctagcgctctgggcaagctgcaggatgtggtgaaccagaacgcccaggctctgaacaccctggtgaagcagctgagcagcaacttcggagccatcagcagcgtgctgaacgacatcctgagcaggctggacaaggtggaagccgaagtgcagatcgacaggctgatcaccggcagactgcagtctctgcagacctacgtgacccagcagctgatcagagccgccgagatcagagctagcgctaatctggccgccaccaagatgagcgagtgcgtgctgggacagagcaagagagtggacttctgcggcaagggctaccacctgatgagctttcctcagagcgctcctcacggagtggtgtttctgcacgtgacctacgtgccagcccaggagaagaacttcaccacagccccagccatttgccacgacggaaaggcccacttccccagagaaggcgtgttcgtgtccaacggcacccattggttcgtgacccagcggaacttctacgagccccagatcatcaccaccgacaacaccttcgtgagcggcaattgcgacgtggtcatcggcatcgtgaacaacaccgtgtacgaccccctgcagccagagctggactccttcaaggaggagctggacaagtacttcaagaaccacaccagcccagacgtggacctgggagacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccttggtacatttggctgggcttcatcgccggcctgatcgcaatcgtcatggtgaccatcatgctctgttgcatgaccagttgctgcagctgcctgaagggttgctgcagttgcgggagttgctgcaagttcgacgaggacgacagcgagccagtgctgaaaggcgtgaagctgcactacacctaa

SEQ ID NO: 9 - DNA, 669 bp, Artificial Sequence
  M protein from Wuhan strain (MN988669)
atggccgactctaacggcaccatcaccgtggaggagctgaagaagctgctcgagcagtggaacctggtcatcggcttcctgttcctcacctggatttgcctgctgcagttcgcctacgccaaccggaaccggttcctgtacatcatcaagctgatcttcctctggctcctctggcccgtgacactggcttgtttcgtgctggcagccgtgtaccggatcaattggatcaccggcggcatcgctatcgctatggcttgtctcgtgggcctgatgtggctgagctacttcatcgccagcttccggctgttcgccagaaccagaagcatgtggagcttcaaccccgagaccaacatcctgctgaacgtgcctctgcacggcacaatcctgaccagacctctgctggagagcgagctggtcatcggagcagtgatcctgagaggccacctgagaatcgccggacatcatctgggacggtgcgacatcaaggacctgcccaaggagatcacagtggccacaagccggaccctgagctactacaagctgggagccagccagagagtggcaggagatagcggattcgccgcctacagcagatacaggatcggcaactacaagctgaacaccgaccacagcagcagcagcgataacatcgccctgctggtgcagtaa

SEQ ID NO: 10 - DNA, 228 bp, Artificial Sequence
  E protein from Wuhan strain (MN988669)
atgtacagcttcgtgtccgaggagaccggaaccctgatcgtgaactccgtgctgctgttcctggcctttgtggtgttcctgctggtgaccctggccattctgacagctctcaggctctgcgcctattgctgcaacatcgtgaacgtgtccctggtgaagcccagcttctacgtgtacagccgcgtgaagaacctgaacagcagcagggtgccagatctgctggtgtaa

SEQ ID NO: 11 - DNA, 1260 bp, Artificial Sequence
  N protein from Wuhan strain (MN988669)
atgagcgacaacggacctcagaaccagcggaacgcccctagaatcacctttggcggccctagcgatagcaccggcagcaatcagaacggcgagagaagcggcgccagatctaagcagaggagacctcagggcctgcctaacaataccgcctcttggttcaccgctctgacacagcacggcaaggaggacctgaagttccctaggggccagggcgtgcctatcaataccaacagcagccccgacgatcagatcggctactacaggagggccaccagaaggatcagaggcggagacggcaagatgaaggacctgagcccccgctggtacttttactatctgggcacaggaccagaggccggactgccttacggagccaacaaggacggcatcatttgggtggccaccgaaggagccctgaacacccctaaggaccacatcggcaccaggaacccagccaataacgccgctatcgtgctgcagctgcctcagggaacaacactgcctaagggcttttacgccgagggcagcagaggaggaagccaggcctctagcagaagcagcagcagaagcaggaacagcagcagaaacagcaccccaggcagcagcagaggaaccagcccagccagaatggcaggaaacggaggagacgcagctctggctctgctgctgctggataggctgaaccagctggagagcaagatgagcggcaagggacagcagcagcagggacagaccgtgaccaagaagagcgccgccgaagcctctaagaagcctagacagaagcggaccgccaccaaggcttacaacgtgacccaggccttcggcagaagaggaccagaacagacccagggcaacttcggcgatcaggaactgatccggcagggcaccgactacaagcattggcctcagatcgcccagtttgctcctagcgccagcgcctttttcggcatgagcaggatcggcatggaagtgacccctagcggaacttggctgacctacaccggagccatcaagctggacgacaaggaccccaacttcaaggaccaggtcatcctgctgaacaagcacatcgacgcctacaagaccttccctcccaccgagcccaagaaggacaagaagaagaaggccgacgagacccaggctctgcctcagaggcagaagaagcagcagaccgtgacactgctgccagcagcagatctggacgacttcagcaagcagctgcagcagagcatgagcagcgccgattctacacaggcctaa

SEQ ID NO: 12 - DNA, 3822 bp, Artificial Sequence
  S glycoprotein with Triple mutations and S1-S2 mutation (Wuhan strain, MN988669)
atgttcgtgttcctcgtgctgctgcccctggtgtctagccagtgcgtgaacctgaccaccagaacccagctgcctccagcctacaccaacagcttcaccaggggagtgtactaccccgacaaggtgttccggagcagcgtgctgcatagcacccaggacctgttcctgcccttcttcagcaacgtgacttggttccacgccatccacgtcagcggaaccaacggcaccaagcgctttgacaacccagtgctgcccttcaacgacggagtgtacttcgccagcaccgagaagagcaacatcatccggggttggatcttcggcaccacactggacagcaagacccagagcctgctgatcgtgaacaacgccaccaacgtcgtgatcaaggtctgcgagttccagttttgcaacgaccccttcctgggcgtgtactaccacaagaacaacaagtcttggatggagagcgagttccgggtgtacagcagcgccaacaattgcaccttcgagtacgtgtcccagcccttcctgatggacctggaaggcaagcagggcaacttcaagaacctgcgggagttcgtgttcaagaacatcgacggctacttcaagatctacagcaagcacacccccatcaacctcgtgagagacctgccacagggcttcagcgctctggaacctctggtggatctgccaatcggcatcaacatcacccggttccagaccctgctggctctgcacagaagctacctgaccccaggcgattcttctagcggctggacagcaggagccgccgcttattacgtgggctatctgcagccccggaccttcctgctgaagtacaacgagaacggcaccatcaccgacgcagtggattgcgctctggatcctctgagcgagaccaagtgcaccctgaagagcttcaccgtggagaagggcatctaccagaccagcaacttccgggtgcagcctacagagagcatcgtgcggttccctaacatcaccaacctctgccccttcggcgaggtgttcaacgccaccagattcgccagcgtctacgcctggaaccggaagcggatcagcaattgcgtggccgactacagcgtgctgtacaacagcgccagcttcagcaccttcaagtgctacggcgtgtcccctaccaagctgaacgatctgtgcttcaccaacgtgtacgccgacagcttcgtgatcaggggcgacgaagtgcgacagatcgccccaggacagacaggaaagatcgccgattacaactacaagctgcccgacgacttcaccggctgcgtgatcgcttggaacagcaacaacctggacagcaaggtcggcggcaactacaactacctgtaccggctgttccggaagagcaacctgaagcccttcgagcgggacatcagcacagagatctaccaggccggcagcacaccttgtaacggcgtggagggcttcaactgctacttccctctgcagagctacggcttccagcctacaaacggcgtgggataccagccttacagggtggtggtgctgagcttcgaactgctgcacgctccagccaccgtctgcggacctaaaaagagcaccaacctggtgaagaacaagtgcgtgaatttcaacttcaacggcctgaccggaaccggcgtgctgaccgagagcaacaagaagttcctgcccttccagcagttcggcagagacatcgcagacaccacagacgccgtgagagatcctcagaccctggagatcctggacatcaccccttgcagctttggcggagtgtccgtgatcaccccaggaaccaacaccagcaatcaggtggccgtgctgtaccaggacgtgaattgcaccgaggtgccagtggccattcacgcagatcagctgacccctacctggagagtgtacagcaccggaagcaacgtgttccagaccagggcaggttgcctgatcggatgcgagcacgtgaacaactgctacgagtgcgacatccccatcggcgccggcatttgcgcctcttaccagacccagaccaacagccctagtggtagcgctagcgtggcctctcagagcatcatctgctacacctgctgcctgggagccgagaacagcgtggcctacagcaacaacagcatcgccatccccaccaacttcaccatcagcgtgaccaccgagattctgcccgtgtctatgaccaagaccagcgtcgactgcaccatgtacatctgcggcgacagcaccgagtgctctaacctgctgctgcagtacggctccttttgcacccagctgaacagggctctgacaggcatcgcagtggagcaggacaagaacacccaggaggtcttcgcccaggtcaagcagatctacaagaccccccctatcaaggacttcggcggctttaacttcagccagatcctgcccgatcccagcaagcctagcaagcggagcttcatcgaggacctgctgttcaacaaggtcaccctggccgacgcaggcttcatcaagcagtacggcgattgcctgggcgacattgccgccagagacctgatttgcgcccagaagttcaacggcctgacagtgctgcctcctctgctgaccgacgagatgatcgcccagtatacaagcgctctgctggcaggaaccatcaccagcggttggacctttggagccggagccgctctgcagatcccttttgccatgcagatggcctaccgcttcaacggaatcggcgtgacccagaacgtgctctacgagaaccagaagctgatcgccaaccagttcaacagcgccatcggcaagatccaggacagcctgagcagcacagctagcgctctgggcaagctgcaggatgtggtgaaccagaacgcccaggctctgaacaccctggtgaagcagctgagcagcaacttcggagccatcagcagcgtgctgaacgacatcctgagcaggctggacaaggtggaagccgaagtgcagatcgacaggctgatcaccggcagactgcagtctctgcagacctacgtgacccagcagctgatcagagccgccgagatcagagctagcgctaatctggccgccaccaagatgagcgagtgcgtgctgggacagagcaagagagtggacttctgcggcaagggctaccacctgatgagctttcctcagagcgctcctcacggagtggtgtttctgcacgtgacctacgtgccagcccaggagaagaacttcaccacagccccagccatttgccacgacggaaaggcccacttccccagagaaggcgtgttcgtgtccaacggcacccattggttcgtgacccagcggaacttctacgagccccagatcatcaccaccgacaacaccttcgtgagcggcaattgcgacgtggtcatcggcatcgtgaacaacaccgtgtacgaccccctgcagccagagctggactccttcaaggaggagctggacaagtacttcaagaaccacaccagcccagacgtggacctgggagacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccttggtacatttggctgggcttcatcgccggcctgatcgcaatcgtcatggtgaccatcatgctctgttgcatgaccagttgctgcagctgcctgaagggttgctgcagttgcgggagttgctgcaagttcgacgaggacgacagcgagccagtgctgaaaggcgtgaagctgcactacacctaa

SEQ ID NO: 13 - DNA, 3813 bp, Artificial Sequence
  S glycoprotein from South Africa strain with mutations (B.1.351)
atgttcgtgttcctcgtgctgctgcccctggtgtctagccagtgcgtgaacctgaccaccagaacccagctgcctccagcctacaccaacagcttcaccaggggagtgtactaccccgacaaggtgttccggagcagcgtgctgcatagcacccaggacctgttcctgcccttcttcagcaacgtgacttggttccacgccatccacgtgtccggaaccaacggcaccaagagattcgccaacccagtgctgcccttcaacgacggagtgtacttcgccagcaccgagaagagcaacatcatccggggttggatcttcggcaccacactggacagcaagacccagagcctgctgatcgtgaacaacgccaccaacgtcgtgatcaaggtctgcgagttccagttttgcaacgaccccttcctgggcgtgtactaccacaagaacaacaagtcttggatggagagcgagttccgggtgtacagcagcgccaacaattgcaccttcgagtacgtgtcccagcccttcctgatggacctggaaggcaagcagggcaacttcaagaacctgcgggagttcgtgttcaagaacatcgacggctacttcaagatctacagcaagcacacccccatcaacctcgtgagaggcctgcctcagggattctcagccctggaacctctggtggatctgccaatcggcatcaacatcacccggttccagaccctgcacagaagctacctgaccccaggcgattcttctagcggctggacagcaggagccgcagcttactacgtgggatacctgcagcccaggaccttcctgctgaagtacaacgagaacggcaccatcaccgacgcagtggattgcgctctggaccctctgagcgagaccaagtgtaccctcaagagcttcaccgtggagaagggcatctaccagaccagcaacttccgggtgcagcctacagagagcatcgtgcggttccccaacatcaccaacctctgccccttcggcgaggtgttcaacgctaccagattcgccagcgtgtacgcttggaaccggaagcggatcagcaattgcgtggccgactacagcgtgctgtacaacagcgccagcttcagcaccttcaagtgctacggcgtgtctcccaccaagctgaacgacctctgcttcaccaacgtgtacgccgacagcttcgtgatccggggagacgaagtgcgccagattgctccaggacagaccggcaacatcgccgactacaactacaagctgcccgacgacttcaccggctgcgtgatcgcctggaacagcaacaacctggacagcaaggtcggcggcaactacaactacctgtaccggctgttccggaagagcaacctgaagcctttcgagcgggacatcagcaccgagatctaccaggcaggcagcacaccttgtaacggagtgaagggcttcaactgctacttccccctgcagagctacggctttcagcctacctacggcgtgggatatcagccctacagggtggtggtgctgagctttgagctgctgcacgccccagctacagtgtgcggacccaagaagagcaccaacctggtgaagaacaagtgcgtgaacttcaacttcaatggcctgaccggcacaggagtgctgacagagagcaacaagaagttcctgcccttccagcagttcggcagggatatcgccgataccaccgacgccgtgagagatcctcagacactggagatcctggacatcaccccttgcagcttcggaggagtgtccgtgatcaccccaggcacaaacaccagcaaccaggtggcagtgctgtaccagggcgtgaattgcaccgaagtgccagtggctatccacgcagaccagctgacccctacttggagggtgtacagcaccggcagcaacgtgttccagaccagagcaggttgtctgatcggagccgagcacgtgaacaacagctacgagtgcgacatccctatcggagccggcatttgcgcctcttaccagacccagaccaactctcctagcggcagcgccagcgtggcctctcagtctatcatcgcctacaccatgagcctgggcgtggaaaacagcgtggcctacagcaacaacagcatcgccatccccaccaacttcaccatcagcgtgaccaccgagatcctgccagtgtccatgaccaagaccagcgtcgactgcaccatgtacatttgcggcgatagcaccgagtgctccaatctgctgctgcagtacggctccttttgcacccagctgaacagagccctgacaggaatcgccgtggagcaggacaagaacacccaggaggtcttcgcccaggtcaagcagatctacaagaccccccccatcaaggacttcggcggcttcaacttcagccagatcctgccagatcccagcaagcctagcaagcggagcttcatcgaggacctgctgttcaacaaggtcacactggccgacgccggctttatcaagcagtacggcgattgcctgggcgacattgcagccagggacctgatttgcgcccagaagttcaacggcctgaccgtgctgcctcctctgctgaccgacgagatgatcgcccagtacacaagcgccctgctggccggaacaatcacaagcggatggacatttggagccggagcagctctgcagatccctttcgccatgcagatggcctaccgcttcaacggaatcggcgtgacccagaacgtgctctacgagaaccagaagctgatcgccaaccagttcaacagcgccatcggcaagatccaggacagcctgtctagcacagccagcgctctgggaaagctgcaggacgtggtgaaccagaacgctcaggccctgaataccctggtgaagcagctgagcagcaacttcggagccatcagcagcgtgctgaacgacatcctgagcaggctggaccctccagaagccgaagtgcagatcgacaggctgatcaccggcagactgcagtctctgcagacctacgtgacccagcagctgatcagagccgcagagatcagagccagcgccaatctggcagctaccaagatgagcgagtgcgtcctgggacagagcaagcgggtggacttttgcggcaagggctaccacctgatgagcttccctcagagcgctcctcacggagtggtgtttctgcacgtgacctacgtgccagcccaggaaaagaacttcaccaccgccccagccatttgccacgacggaaaggcccactttcctagagagggcgtgttcgtgtccaacggcacccattggttcgtgacccagcggaacttctacgagccccagatcatcaccaccgacaacaccttcgtgagcggcaattgcgacgtggtcatcggcatcgtgaacaacaccgtgtacgaccccctgcagccagagctggactccttcaaggaggagctggacaagtacttcaagaaccacaccagcccagacgtggacctgggcgatatcagcggcatcaacgccagcgtggtgaacatccagaaggagatcgacaggctgaacgaggtggccaagaacctgaacgagagcctgatcgacctgcaggagctgggcaagtacgagcagtacatcaagtggccttggtacatttggctgggcttcatcgccggcctgatcgccatcgtcatggtcaccatcatgctctgctgcatgaccagttgctgctcttgcctgaagggttgctgcagctgcgggtcttgttgcaagttcgacgaggacgacagcgagccagtgctgaagggcgtgaagctgcactacacctaa

SEQ ID NO: 14 - DNA, 3822 bp, Artificial Sequence
  S glycoprotein from India strain with mutations ((B .1. 617)
atgttcgtgttcctcgtgctgctgcccctcgttagttctcaatgcgtgaacctgaccacgcggacccagctgccacccgcctataccaactcatttactcgaggggtgtactatcccgataaggtgtttcgatcatctgtactccacagcacccaagatctgtttctcccattcttcagtaacgtgacctggtttcacgccattcacgtgagtggcactaacggcacaaaacgcttcgacaatccggtgctgcctttcaacgacggcgtatacttcgcatcaatagagaaaagcaatatcatccggggttggatattcggcacaaccctggatagcaaaacacaaagtctgctcatcgtcaacaatgcgacgaacgtagtgatcaaggtgtgcgagttccagttctgcaacgaccccttcctggacgtgtactatcataagaataataagtcctggatgaagtcagaatttcgggtgtactcaagcgctaacaactgtaccttcgagtacgtgtcacaaccattcctcatggatctcgagggcaagcaaggaaactttaagaacttgagagagttcgttttcaagaacatcgacggctacttcaagatttacagtaaacatacaccaatcaacctggttagagacctgccacaaggcttctctgccctcgagcccctcgtcgacctgcccatcgggatcaatattacgcgtttccagacactcctggccctgcacaggtcatacctcacacccggcgactcatcatccgggtggactgcaggagccgctgcctactacgtaggatacctgcagccccgcaccttcctgctgaagtacaacgagaacggcactataaccgacgccgtggattgcgctctcgatcccctttctgagacgaaatgcactctcaagtcatttaccgtcgagaagggcatttaccagaccagcaatttcagggttcagcctactgagtcaatcgtgcgattcccaaacatcacgaatctgtgtccgttcggggaagtcttcaatgcgactcggttcgccagcgtgtacgcctggaatagaaaacgtattagtaattgcgtggcagactactccgtgctttacaacagcgctagtttctctacattcaaatgctacggggtaagtcccacaaagttgaacgacttgtgtttcacaaacgtatacgcggacagcttcgtgatccgtggcgacgaggtgcgccagattgcccccggtcagacagggaaaatagccgactacaactacaagcttcccgacgacttcacgggttgtgtgattgcctggaactcaaataacctcgacagcaaagtcggtgggaactacaactataggtacaggctgttccgcaaatcaaacctgaagcccttcgaacgcgacatcagtaccgagatataccaagctggatctactccatgcaacggcgtccagggcttcaactgctatttccccctgcagagttacgggtttcagcctacaaacggagtgggatatcagccttatcgggtggtcgtcctgagtttcgagctgcttcacgctcctgcgaccgtctgcggccccaagaaatcaacgaaccttgtaaagaataagtgcgttaactttaattttaacggcctgaccgggactggggtgttgacagaatccaataagaaattccttccatttcagcagttcggtagggatatagcagataccaccgacgccgtgagggaccctcaaacgctcgaaatcctcgatatcaccccctgctcattcggcggggtttccgtcatcacccccgggaccaacacctcaaatcaagtggccgtgctgtaccaaggggtcaattgtactgaggttccggtggcaatccacgccgaccagctgaccccaacctggagagtgtactccactggcagtaacgtcttccagacccgcgctggttgcctcatcggcgccgagcacgtgaataattcctacgaatgcgatatccctatcggagccgggatctgtgcatcataccaaacgcaaaccaacagcagaagcggcagtgcatccgtggcatctcagagcataatcgcgtatacaatgagtttgggcgccgagaactctgtcgcatattccaacaattccatcgcgatccctacgaacttcaccatctctgtcacaaccgagatactccctgtatcaatgactaaaactagtgtggactgcaccatgtatatctgcggcgacagcaccgagtgttctaacctgctgctgcagtacggaagcttctgcactcagttgaatcgagccctgacgggcattgccgtggagcaggataagaatacgcaggaagtcttcgctcaggtgaagcagatctataagactccccctatcaaggacttcggcggcttcaacttctctcagattttgcccgacccttccaagccttcaaaacgcagcttcatcgaggacctgttgtttaataaggttaccctggccgacgccggatttattaagcagtacggggactgtctcggggacatcgcagcaagggatctgatatgcgcccagaaattcaatgggttgacagtacttcccccgctgcttactgacgagatgatcgcccagtatacgagcgcgctcctggccgggacgattacatcagggtggactttcggcgctggggcagccctgcagattcccttcgccatgcagatggcctaccgcttcaacggcatcggcgtgacccaaaacgtgctgtacgaaaatcagaagctgatcgcaaatcagttcaactccgccatagggaagatccaggatagcttgagctcaaccgcctctgctctcgggaagctccaggacgtcgtgaatcagaacgctcaggccctcaataccctggtcaagcagctcagtagcaacttcggcgctatctcttctgtgttgaacgacattctgagcaggctggatccaccggaagcagaggtccagatagaccgactgattactggaaggctgcagagccttcaaacttacgtaacccagcagcttatcagggcagccgagataagggccagcgccaacttggccgcgacgaagatgtctgaatgcgttttgggccagtctaagagggtggacttctgcgggaaagggtaccacttgatgtcatttccccaaagcgccccacacggagtggtgtttcttcacgtcacctacgtgcccgctcacgagaagaattttaccacagcgcccgctatatgccacgacggtaaggcccatttccccagagagggcgtgttcgtgagcaacgggactcattggttcgtcacgcagcgcaacttctacgagccccagattatcaccactgataatactttcgtcagcggcaattgcgacgtggttattggcatcgtaaataatactgtgtacgacccgctgcagcccgagctcgattcctttaaagaagaactcgacaagtacttcaagaaccacactagccccgacgtcgacctcggagatatttccgggataaacgccagcgtcgtgaatatacagaaggagatcgatagactgaacgaagtcgctaagaacctgaacgagagccttattgacctgcaggagctcggtaaatacgaacaatacattaagtggccttggtatatatggctcggcttcatcgcgggactgatcgctatcgtcatggttactatcatgctgtgttgcatgacttcatgttgctcatgccttaaaggatgctgctcctgcgggagctgttgtaagttcgacgaggatgattcagagccggtattgaaaggggtaaagctccactatacttga

SEQ ID NO: 15 - protein, 7 aa, Artificial Sequence
  A653C
LIGAEHV

SEQ ID NO: 16 - protein, 7 aa, Artificial Sequence
  A694C
SIIAYTM

SEQ ID NO: 17 - protein, 7 aa, Artificial Sequence
  S659C
VNNSYEC

SEQ ID NO: 18 - protein, 7 aa, Artificial Sequence
  S698C
YTMSLGA

SEQ ID NO: 19 - protein, 7 aa, Artificial Sequence
  C662C
SYECDIP

SEQ ID NO: 20 - protein, 7 aa, Artificial Sequence
  M697C
AYTMSLG

SEQ ID NO: 21 - protein, 7 aa, Artificial Sequence
  A570C
RDIADTT

SEQ ID NO: 22 - protein, 7 aa, Artificial Sequence
  V963C
NTLVKQL

SEQ ID NO: 23 - protein, 7 aa, Artificial Sequence
  D571C
DIADTTD

SEQ ID NO: 24 - protein, 7 aa, Artificial Sequence
  S967C
KQLSSNF

SEQ ID NO: 25 - protein, 7 aa, Artificial Sequence
  K558C
SNKKFLP

SEQ ID NO: 26 - protein, 7 aa, Artificial Sequence
  N282C
YNENGTI

SEQ ID NO: 27 - protein, 7 aa, Artificial Sequence
  P862C
TVLPPLL

SEQ ID NO: 28 - protein, 7 aa, Artificial Sequence
  A668C
PIGAGIC

SEQ ID NO: 29 - protein, 7 aa, Artificial Sequence
  Y707C
SVAYSNN

SEQ ID NO: 30 - protein, 7 aa, Artificial Sequence
  T883C
GTITSGW

SEQ ID NO: 31 - protein, 7 aa, Artificial Sequence
  V705C
ENSVAYS

(19) United States
(12) Patent Application Publication
(10) Pub. No.: US 2023/0321219 A1
(43) Pub. Date: Oct. 12, 2023

(54) SEVERE ACUTE RESPIRATORY SYNDROME CORONAVIRUS [SARS-CoV-2]-VIRUS-LIKE PARTICLE [VLP] VACCINE: COMPOSITIONS, DELIVERY STRATEGIES, METHODS AND USES

(71) Applicant: TECHNOVAX, INC., Elmsford, NY (US)

(72) Inventors: Jose M. GALARZA, Kinnelon, NJ (US); Ke WEN, Harrison, NY (US); Ryan MAZBOUDI, Pleasantville, NY (US); Matthew D. RESCH, Rochester, NY (US)

(21) Appl. No.: 18/041,883

(22) PCT Filed: Aug. 17, 2021
(86) PCT No.: PCT/US2021/046353

§ 371 Date: Feb. 16, 2023

Related U.S. Application Data
(60) Provisional application No. 63/066,617, filed on Aug. 17, 2020.

(51) Int. Cl. A61K 39/215; A61P 31/14; C12N 7/00; C12N 15/85

(52) U.S. Cl. CPC A61K 39/215; A61P 31/14; A61K 2039/5258; C12N 15/85; C12N 7/00

(57) ABSTRACT

The present application relates to SARS-CoV-2 virus-like particles (VLP) and related plasmids, compositions, and methods. The VLP can comprise a modified spike glycoprotein, a matrix protein, a nucleoprotein N and an envelope protein of SARS-CoV-2, where the modified spike glycoprotein comprises an S1 domain and an S2 domain, and includes one or more modifications. These modifications can include: linking the S1 and S2 domains via generation of disulfides bonds between the S1 and S2 domains; linking intra-polypeptide and inter-polypeptide S2 helices of the S2 domain; and substitution of one or more non-cysteine residues with a cysteine residue to generate one or more disulfide bonds. The modifications can stabilize a prefusion conformation of the spike glycoprotein and prohibit a transition to a post-fusion structure.

### CROSS REFERENCE TO RELATED APPLICATIONS

This application is a U.S. National Phase Application under 35 U.S.C. § 371 of International Patent Application No. PCT/US2021/046353, filed on Aug. 17, 2021, which claims priority from U.S. Provisional Application No. 63/066,617, filed on Aug. 17, 2020, all of which are incorporated by reference, as if expressly set forth in their respective entireties herein.

### SEQUENCE LISTING

The present application contains a Sequence Listing which has been submitted electronically in ASCII format and is hereby incorporated by reference in its entirety. Said ASCII copy, created on Feb. 16, 2023 is named 10050-010184-US1 and is 76.1 KB in size.

### TECHNICAL FIELD

The present application relates to virus-like particles (VLPs), compositions comprising virus-like particles (VLPs), and methods of making or delivery of such VLPs. More specifically, the present application relates to VLPs of viruses of the Coronaviridae family.

### BACKGROUND

The SARS-CoV-2 coronavirus, in many cases, causes a severe condition termed coronavirus-19 disease (COVID-19). The recently emerged SARS-CoV-2 virus has spread around the world causing a devastating pandemic inflicting human suffering and economic hardship.

The rapid emergence of this novel virus, with which the human immune system had no experience, left global public health systems scrambling to develop countermeasures to combat the pandemic and treat patient suffering. The most important defense against this agent will be the development of a safe and effective vaccine able to elicit protective immunity in all ages. Reducing the susceptible pool of possible patients and therefore virus transmission, has the potential to stem the tide of this pandemic.

The etiological agent of COVID-19, SARS-CoV-2 is a coronavirus that belongs to the family Coronaviridae in the order Nidovirales. Members of this distinct order of viruses are composed of a membrane-envelope, a large non-segmented positive sense RNA genome, and a nucleocapsid that is packaged within virions of a diverse architecture. Based on phylogenetic analysis, the Coronvirinae subfamily is classified into four genera (Alphacoronavirus, Betacoronavirus, Gammacoronavirus and Deltacoronavius), and the betacoronavirus genus is further divided into four lineages (A, B, C and D), which include viruses isolated from mammalian species and birds. The severe acute respiratory syndrome-related coronavirus-2 (SARS-CoV-2) belongs to the genus betacoronavirus, which also includes human coronavirus OC43, severe acute respiratory syndrome coronavirus (SARS-CoV), Middle East respiratory syndrome coronavirus (MERS-CoV) as well as coronaviruses affecting other species. The four endemic human coronaviruses HCoV-229E, -OC43, -NL63 and HKU1 cause lower and upper respiratory disease in adults and children that may range from the common cold to pneumonia, but in most people, resulting symptoms are generally mild. The other human coronaviruses, however, can cause severe respiratory illness and they include SARS-CoV first identified in China in 2003, and MERS-CoV, first identified in Saudi Arabia in 2014.

The newly emerged SARS-CoV-2 was first identified in patients with pneumonia in the city of Wuhan, China and from there it began spreading, initially to close contacts of patients and healthcare workers then eventually into the general community, and shortly after to other countries around the world. Its origin has yet to be determined, but results from comparative genomic analysis provides some perspective as to how this virus could have jumped species boundaries to infect humans. The unfortunate highly efficient transmissibility of this virus has allowed it to cause significant global morbidity and mortality, overwhelming health systems and disrupting social interaction and economic activity to a degree not seen in recent times. Creating a safe and effective vaccine, therefore, is vitally important not only to protect lives and fight the pandemic but also to create herd immunity, reduce virus circulation and restore normalcy world around. To this end, the present application describes a virus-like particle (VLP) technology suitable for a SARS-CoV-2 vaccine as well as other coronavirus vaccines and their compositions, methods and field of use.

### SUMMARY

Described herein is a SARS-CoV-2 virus-like particle (VLP). The SARS-CoV-2 VLP includes a modified spike (S) glycoprotein of SARS-CoV-2, a matrix (M) protein of SARS-CoV-2, and an envelope (E) protein of SARS-CoV-2. The modified S glycoprotein comprises an S1 domain and an S2 domain. The modified S glycoprotein also includes at least one of the following modifications: (i) linking the S1 and S2 domains via generation of disulfides bonds between the S1 and S2 domains; (ii) linking intra-polypeptide and inter-polypeptide S2 helices of the S2 domain; and (iii) substitution of one or more non-cysteine residues with a cysteine residue to generate one or more disulfide bonds. The modifications to the S glycoprotein (i) stabilize a prefusion conformation of the S glycoprotein and/or (ii) prohibit a transition to a post-fusion structure.

In another aspect, the linking of the S1 and S2 domains results from one or more of the following pairs of cysteine substitutions: (i) A653C at the S1 domain and A694C at the S2 domain; (ii) S659C at the S1 domain and S698C at the S2 domain; and (iii) C662C at the S1 domain and M697C at the S2 domain.

In another aspect, the linking of intra-polypeptide and inter-polypeptide S2 helices of the S2 domain to one another can results from one or more of the following pairs of cysteine substitutions: (i) Y707C and T883C at the S2 domain; and (ii) V705C and T883C at the S2 domain.

In another aspect, the substitution of one or more non-cysteine residues with a cysteine residue generates one or more disulfide bonds that prohibit the spike receptor binding domain (RBD) from a conformational change which includes one or more of the following substitutions: (i) A570C at the S1 domain and V963C at the S2 domain; (ii) D571C at the S1 domain and S967C at the S2 domain; and (iii) K558C at the S1 domain and N282C at the S2 domain.

In another aspect, the modifications to the S glycoprotein further include a P862C substitution at the S2 domain and A668C at the S1 domain, where these substitutions result in the locking of the S1 domain of one polypeptide chain to the S2 of another polypeptide chain, resulting in the stabilization of a prefusion conformation of the modified S glycoprotein.

In another aspect, the SARS-CoV-2 VLP further comprises an additional modification to the S glycoprotein, where the additional modification comprises replacing one or more domains of the SARS-CoV-2 S glycoprotein with analogous portions from one or more other coronaviruses to produce a chimeric or mosaic S glycoprotein.

In another aspect, the modified S glycoprotein is further coexpressed with the matrix (M) protein or the matrix M and envelope (E) or the matrix (M), the envelope (E) and a nucleocapsid (N) protein of SARS-CoV-2.

In another aspect, the modifications to the S glycoprotein include at least one of the following pairs of cysteine substitutions: (i) A653C at the S1 domain and A694C at the S2 domain; (ii) S659C at the S1 domain and S698C at the S2 domain; (iii) C662C at the S1 domain and M697C at the S2 domain; (iv) V705C and T883C at the S2 domain; (v) A570C at the S1 domain and V963C at the S2 domain; (vi) D571C at the S1 domain and S967C at the S2 domain; (vii) Y707C and T883C at the S2 domain; and (vii) K558C at the S1 domain and N282C at the S2 domain.

In one aspect, the SARS-CoV-2 VLP further comprising at least one or more of the following mutations:
(i) one or more amino acid residues at position 681-684 in an alpha variant;
(ii) one or more amino acid residues at position 679-682 in a beta variant;
(iii) one or more amino acid residues at position 682-685 in a delta variant;
(iv) one or more amino acid residues at position 814-815;
(v) one or more amino acid residues at position 983-984 in a beta variant;
(vi) one or more amino acid residues at position 986-987 in a delta variant,

and wherein for (i) to (iii), the one or more mutations are from RRAR to SGSA, and wherein for (iv) the one or more mutations are from KR to SG, and wherein for (v) to (vi) the one or more mutations are from KV to PP.

In another aspect, the SARS-CoV-2 VLP is suitable for the preparation of a SARS-CoV-2 vaccine.

Also described herein is an expression plasmid comprising genes encoding coronavirus structural and surface proteins, wherein the expression plasmid is suitable for the assembly of the SARS-CoV-2 VLP. The expression plasmid comprises optimized genes encoding a modified SARS-CoV-2 spike (S) glycoprotein, a SARS-CoV-2 matrix (M) protein, and a SARS-CoV-2 spike envelope (E) protein.

In another aspect, the expression plasmid further comprises optimized genes encoding a nucleocapsid (N) protein of SARS-CoV-2.

Also described herein is a method for producing a SARS-CoV-2 VLP, the method comprising introducing into a host cell at least one expression plasmid suitable for the assembly of the SARS-CoV-2 VLP, where the expression plasmid is introduced into the host cell under conditions such that the host cell produces the SARS-CoV-2 VLP.

In another aspect, the host cell is a eukaryotic cell. In a further aspect, the eukaryotic cell is a mammalian cell. In a further aspect, the eukaryotic cell is stably modified to continuously produce a VLP vaccine, such as a SARS-CoV-2 VLP vaccine.

Also described herein is an immunogenic composition comprising at least one SARS-CoV-2 VLP of the present application.

Also described herein is a method of generating an immune response to one or more coronaviruses in a subject. The method comprises administering an effective amount of the immunogenic composition of the present application to the subject. In another aspect, the immunogenic composition is administered nasally, mucosally or parenterally. In another aspect of the method, the subject is a human. In another aspect, the immune response vaccinates the subject against one or more coronaviruses. In a further aspect, the immune response vaccinates the subject against SARS-CoV-2.

### BRIEF DESCRIPTION OF THE DRAWINGS

FIG. 1. Structural Domains of SARS-CoV-2 Spike Surface Glycoprotein. Diagram of SARS-CoV-2 spike (S) protein, the domains in the primary structure are colored and labeled.

FIG. 2. Expression Plasmid for VLP Production. Schematic of one of the expression plasmids carrying the SARS CoV-2 genes (S, M, E and N) used for VLP assembly in accordance with one or more embodiments.

FIG. 3. Western Blot Analysis of SARS-CoV-2 Virus-Like Particles (CoV-2 VLPs) Purified by Chromatography. SARS-CoV-2 VLPs were purified by anion-exchange chromatography and eluted fractions from the column analyzed via Western blot using an anti-spike specific antibody. The spike glycoprotein expressed on the surface of the particles contains modifications in one of the cleavage sites and disulfide bridges that stabilized the molecule for the best display of neutralizing antigenic determinants. The detection of a full-length size of the spike reflects these modifications. WW: Molecular weight markers; SM: Starting material; FT: Flow through; Number 9 to 15 elution fractions.

FIG. 4. Dot Blot Analysis of SARS-CoV-2 Matrix. SARS-CoV-2 VLPs were purified by anion-exchange chromatography and eluted fractions from the column analyzed via dot blot using an anti-matrix specific antibody. Numbers 9 to 15 represent the elution fractions from the chromatography column.

FIG. 5. Electron Micrograph of SARS-CoV-2 Virus-Like Particles (CoV-2 VLPs). SARS-CoV-2 virus-like particles (VLPs) were produced in suspension culture of mammalian cells using the virion structural proteins together with a stabilized spike surface glycoprotein. Released VLPs were purified via chromatography and examined by negative staining electron microscopy. Particles show a moderate level of pleomorphism and typical club like projections of the spike protein that characterizes the coronavirus group. Bar 1 um: 1 micron.

FIG. 6. Electron Micrograph of SARS-CoV-2 Virus-Like Particles (CoV-2 VLPs)-Negative Staining and Immunogold Labelling. Samples of purified SARS-CoV-2 virus-like particles (VLPs) were examined by electron microscopy following negative staining with phospho-tungstic acid in panel A; or immunogold labeled with an anti-spike specific antibody as primary (rabbit) and a gold bead conjugated anti-rabbit as secondary in panel B. The black dots (panel B, gold beads) show that the spikes projections seen on the surface of the particles (A) are indeed composed of the SARS-CoV-2 spike glycoprotein.

FIG. 7. Structural conformations of the S protein. The coloring scheme is described in the table top right cell. The engineered disulfides are shown as yellow stick models.

FIG. 8. Structural Domains and Modifications of the SARS-CoV-2 Spike Surface Glycoprotein. Schematics of the SARS-CoV-2 spike protein native primary structure (top panel), and examples of mutations and modifications (lower panel), 1. Mutations and changes in the cleavage sites; 2. Introduction of cysteine changes for the formation of disulfide bridges; and 3. Domains swapping (represented by color changes). SS, signal sequence; NTD, N-terminal domain; RBD, receptor binding domain; SD1 and SD2, subdomain 1 and 2; S1/S2 subunits boundary cleaved in many coronaviruses e.g. SARS-CoV-2), which remain non covalently bound in the prefusion conformation; S2′ host protease cleavage site upstream from the fusion peptide, FP; HR1, heptad repeat 1; CH, Central helix; CD, Connector domain; HR2, Heptad repeat 2; TM, Transmembrane domain; CT, Cytoplasmic tail.

FIG. 9. Flow chart outlining the purification stages of the SARS-CoV-2 VLP vaccine following production in suspension culture of mammalian cells in accordance with one or more embodiments. The process produces a highly purified final vaccine product.

FIGS. 10A-10D. ELISA Antibody Titers Specific for the SARS-CoV-2 Spike Protein. Mice were immunized twice using a prime and booster regimen with COVID-19 vaccine formulated with three different adjuvants (groups 1-3; FIGS. 10A-10C) or PBS control (group 4;

FIG. 10D). Serum samples were collected three weeks after the booster dose and specific antibodies titers against the Wuhan spike protein measured via ELISA. The COVID-19 VLP vaccine elicited the production of significant levels of anti-Spike antibody titers as compared to the pre-immunization control serum (gray dots-pre-immunization bleed). Statistical significance was determined by a two-way ANOVA with a Tukey post hoc test. Asterisks represent significance between Female (Covid VLP Vaccine) and Mouse Serum Samples of Pre-Immunization Bleed control (Pre-Bleed). Pound symbols represent significance between Male (Covid VLP Vaccine) and Mouse Serum Samples of Pre-Immunization Bleed control *p<0.05, **p<0.01, ***p<0.001, ****p<0.0001; #p<0.05, ##p<0.01, ###p<0.001, ####p<0.0001.

### DETAILED DESCRIPTION

The present disclosure describes the formation of SARS-CoV-2 virus-like particles (VLPs) using the structural proteins of coronavirus and their modified version in order not only to improve production and stabilize the structure but also to enhance their immunological properties for better performance when used as vaccine. It also describes methods of production, e.g. transient or stable production in mammalian cells or any other eukaryotic cell expression system (e.g. insect cells/baculovirus expression system, yeast and others), forms of delivery such as premade purified VLPs or utilizing a viral vector (e.g., adenovirus) that expresses the proteins required for VLP formation after administration to humans, an example of a vector is adenovirus 55 (AdV55), or other viral vector such as measles virus, vesicular stomatitis virus (VSV), reoviruses, retroviruses, alphavirus, herpes, picornavirus, etc. or nucleic acid based vaccine approaches such as DNA (plasmid) or mRNA based vaccines. Further described are modifications to the surface spike in order to create more potent immunogens or multivalent vaccine compositions. Besides its vaccine use, this VLP technology has multiple uses and field of applications such as diagnostics, therapeutics, delivery platform, etc.

In one or more embodiments, the present application relates to virus-like particles (VLPs) of viruses of the Coronaviridae family [e.g., severe acute respiratory syndrome coronavirus 2 (SARS-CoV-2) its different genotypes, serotypes and antigenic variants or other members of the family].

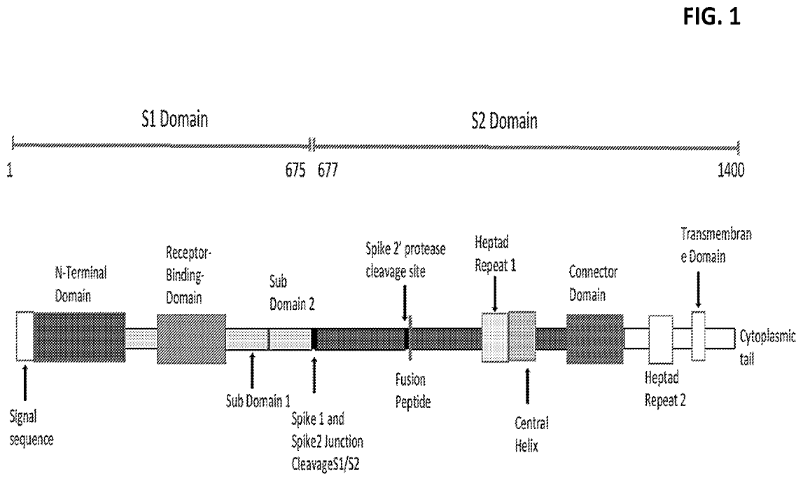

The coronavirus virion consists of a cell-derived lipid bilayer constituting the viral envelope which encases a helical structure resulting from the association of the single stranded positive-sense non-segmented RNA genome with the nucleocapsid protein (N). The virion envelope of SARS-CoV-2 contains three virus-encoded proteins, the type I surface glycoprotein (S), the membrane embedded matrix protein (M), and a small protein found in the surface designated envelope (E). The typical surface spikes of coronaviruses are composed of trimers of the S molecule and each monomer contains a transmembrane anchoring domain, a very large ectodomain and a small intracellular tail. The spike is a multifunctional glycoprotein molecule that recognizes the human host cell receptor, angiotensin-converting enzyme 2 (hACE-2), undergoes a proteases cleavage by a cellular protease to expose the fusion peptides and subsequently mediates membrane fusion enabling virus entry. These functions of the spike protein S are performed by two distinct domains of the external portion of the molecule. The distal top portion S1 domain mediates receptor binding, whereas the envelope anchored S2 domain promotes fusion of the viral and cell membranes enabling virus entry into the host cells, FIG. 1. Given its prominence on the virion surface, the spike S is the primary target of the immune system. Therefore, elicitation of a potent neutralizing antibody response targeting the S protein will provide the best protection against infection with SARS-CoV-2. Considering that S may display alternative conformations (e.g., prefusion and postfusion), its paramount to present to the immune system not only a stable conformation of the immunogen but also one that exhibits the most potent antigenic determinants.

In accordance with one or more embodiments described herein, the VLPs of the present application introduce changes to the S molecule in order to stabilize its conformation in the prefusion state and display the most potent neutralizing epitopes. Assembly of COVID-19 virus-like particles (VLPs) is accomplished by co-expressing S together with the matrix M, envelope E, and nucleoprotein N or S and M, or S, M and E in which S, M and E may contain changes not only to stabilize S and ensure best neutralizing epitopes display but also to enhance particle assembly and yield. Modification of S may encompass mutations, deletions and/or substitutions within the S1/S2 cleavage site with or without analogous changes to the S2′ cleavage site. In certain embodiments, the S1/S2 cleavage site is mutated. In certain embodiments, the Spike 2 or S2′ cleavage site is mutated. In certain embodiments, one or more mutations at the S1/S2 cleavage site in the Wuhan, original Alpha variant is at one or more amino acid residues at position 681-684. In one embodiment, the one or more amino acid substitutions are from RRAR->SGSA. In certain embodiments, one or more mutations at the S1/S2 cleavage site in the South African, Beta variant is at one or more amino acid residues at position 679-682. In one embodiment, the amino acid substitutions are from RRAR->SGSA. In certain embodiments, one or more mutations at the S1/S2 cleavage site in the Indian, Delta variant is at one or more amino acid residues at position 682-685. In one embodiment, the amino acid substitutions are from RRAR->SGSA. In one embodiment, one or more mutations at the S2′ cleavage site is at one or more amino acid residues at position 814-815. In one embodiment, the amino acid substitutions are from KR->SG. In certain embodiment, additional mutations or modifications are present at the spike protein. In one embodiment, the additional mutations or modifications is at one or more amino acid residues at position 983-984 in the Beta variant. In one embodiment, the one or more mutations are from KV->PP. In one embodiment, the additional mutations or modifications are at one or more amino acid residues at position 986-987 in the Delta variant. In one embodiment, the one or more mutation are from KV->PP.

In certain embodiments, one or more amino acid residues at positions 675, 676 and 677 are mutated. In certain embodiments, one or more amino acid residues at positions 682, 683, 684 and 685 are mutated. In certain embodiments, one or more amino acid residues at positions 707-800. Also, S ectodomain specific changes may consist of mutations or substitutions of adjacent amino acid residues within structural domain such as cysteine substitutions which form disulfide bonds (disulfide bridges) between these neighboring residues locking/stapling/or fixing the molecule in a particular conformation preventing further structural molecular changes.

Additional modifications may comprise domain swapping in which, for example, a portion of the SARS-CoV-2 spike such as the receptor binding domain (RBD) or a complete S1 domain or portion thereof all are exchanged with homologous regions from antigenic variants or another coronavirus of the coronaviridae family. This chimera or hybrid molecules may allow for the design of multivalent, broadly protective, universal or pancoronavirus vaccines. Similarly, the exchange of the transmembrane domain or endodomain of the spike enables contact optimization with homologous or heterologous morphogenesis factor such as the matrix M, the envelope E or nucleocapsid N proteins.

The present application is also directed to compositions comprising the VLPs, and methods of making or delivery of such VLPs as premade or utilizing a vector (e.g. adenovirus) that expresses the genes necessary to assemble the VLP within immunized people and using these premade or vector delivered VLPs, including the creation and production of virus-like particle (VLP) based vaccines (e.g., monovalent, polyvalent, single particle universal or polyvalent, single particle mosaic or modified chimeric compositions). The present application also relates to the use of the present VLPs and VLP-based compositions for therapeutic delivery [(e.g., small molecules, nucleic acids, antibodies, enzymes (nanocarriers, nanobodies)] diagnostic, immunomodulatory functions and therapeutic indications.

In particular, in accordance with one or more embodiments, the present disclosure includes strategies and methods used for the modification and stabilization of the major Coronavirus surface antigen, the spike glycoprotein S, which is a metastable protein that transitions from a prefusion to a postfusion conformation in order to perform its functions of receptor binding and membrane fusion and thus mediate viral entry into host cells. Furthermore, several coronaviruses affect human including epidemic strains such as human coronavirus 229E, human coronavirus NL63, human coronavirus OC43, human coronavirus HKU1, in addition to the current pandemic SARS-CoV-2 and the less frequent but highly pathogenic SARS-CoV and Middle East respiratory syndrome virus (MERS). In one more embodiments, the technology described herein is suitable not only for each of these targets but also for the development of multivalent, universal or pancoronavirus vaccines that are able to protect humans against infection with one or more coronavirus types, clades or antigenic variants of the coronviridae family. Also described herein are the coronavirus proteins required for VLP assembly and production methods (e.g. secretion systems) that produce VLPs that display certain antigenic configurations or modifications of the spike (S) major surface glycoprotein. These VLPs feature stabilized conformations of the spike or chimeric epitopes of the receptor binding domain (RBD) relevant for the generation of an enhanced neutralizing immune response to one or more coronaviruses. Single particle monovalent, bivalent, multivalent, universal or chimeric (e.g., different coronaviruses and genotypes such as SARS-CoV-2, epidemic human coronavirus 229E, NL63, OC63, HKU1, SARS-CoV, and MERS). VLPs are assembled and used to formulate vaccine compositions, which allows for immunization and subsequent protection against one or more coronaviruses or antigenically distinct spikes (e.g. SARS-CoV-2, 229E, NL63, OC43, HKU1, MERS etc.) VLPs with stabilized, modified or reengineered spike glycoprotein monomers enables the linking/conjugation of different molecular entities to the external surface of the particle (small or large molecular entities) or the encapsidation of such molecular entities within the structure of the VLPs using alternative packaging methods.

Furthermore, VLPs are also used for the diagnosis of infection or for therapeutic indications. VLP vaccines can be produced via transient transfection of suspension culture of eukaryotic cells or suspension culture of stably transfected cells that constitutively produce the VLPs, which are released into the culture medium. After purification, concentration, and formulation the vaccine can be administered by any suitable route, for example, via either mucosal or parenteral routes, and induce an immune response able to protect against any or all coronaviruses, antigenic variants, etc. VLPs comprising therapeutics, immunomodulatory functions and diagnostic application are also provided.

### Definitions

As used in this specification and the appended claims, the singular forms “a,” “an” and “the” include plural references unless the content clearly dictates otherwise. Thus, for example, reference to “a VLP” can include a mixture of two or more such VLPs.

As used herein the term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

An “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a cytotoxic T lymphocyte (CTL) epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts which produce the antigens.

As use herein, the term “antigenic formulation” or “antigenic composition” refers to a preparation which, when administered to a vertebrate, e.g. a mammal, will induce an immune response.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein, “disulfides bonds” or “disulfide bridges” refer to a disulfide that links two cysteine residues in a peptide or protein. They are a type of covalent bond in protein structures, and usually help to maintain or improve structural stability of proteins. A disulfide bridge between two cysteine residues can be formed by oxidation, for example.

As used herein an “effective dose” generally refers to that amount of VLPs of the invention sufficient to induce immunity, to prevent and/or ameliorate an infection or to reduce at least one symptom of an infection and/or to enhance the efficacy of another dose of a VLP. An effective dose may refer to the amount of VLPs sufficient to delay or minimize the onset of an infection. An effective dose may also refer to the amount of VLPs that provides a therapeutic benefit in the treatment or management of an infection. Further, an effective dose is the amount with respect to VLPs of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “effective amount” refers to an amount of VLPs necessary or sufficient to realize a desired biologic effect. An effective amount of the composition would be the amount that achieves a selected result, and such an amount could be determined as a matter of routine experimentation by a person skilled in the art. For example, an effective amount for preventing, treating and/or ameliorating an infection could be that amount necessary to cause activation of the immune system, resulting in the development of an antigen specific immune response upon exposure to VLPs of the invention. The term is also synonymous with “sufficient amount.”

As used herein, “glycoproteins” refers are proteins which contain oligosaccharide chains (glycans) covalently attached to amino acid side-chains. A spike (S) glycoprotein is a glycoprotein that protrudes from the envelope of some viruses (e.g., coronaviruses) and facilitates entry of the virion into a host cell by binding to a receptor on the surface of a host cell followed by receptor mediated endocytosis and subsequent fusion of the viral and host cell membranes.

An “immunogenic composition” is a composition that comprises an antigenic molecule where administration of the composition to a subject results in the development in the subject of a humoral and/or a cellular immune response to the antigenic molecule of interest.

An “immunological response” or “immune response” to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T lymphocytes (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γΔ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term “multivalent” refers to VLPs which have multiple antigenic proteins against multiple types or strains of infectious agents or alternative conformations of the same antigen/protein (metastable), which naturally transition from one conformation to the next, but in the context of a vaccine formulation may contain stabilized (fixed) form of one conformation or both.

A “nucleic acid” molecule can include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

“Operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

By “pharmaceutically acceptable” or “pharmacologically acceptable” is meant a material which is not biologically or otherwise undesirable, i.e., the material may be administered to an individual in a formulation or composition without causing any unacceptable biological effects or interacting in a deleterious manner with any of the components of the composition in which it is contained.

As used herein “pre-fusion”, “prefusion” or “prefusion conformation” refers to the conformation of the spike (S) glycoprotein of SARS-CoV-2 virus before fusion of the viral and host membrane. In contrast, “postfusion”, “post-fusion”, or “post-fusion conformation” refers to the conformation of the S glycoprotein after fusion of the viral and host membrane. This change in conformation of the S glycoprotein from the prefusion to the postfusion conformation initiates infection.

As used herein the term “protective immunity”, “protective immune response” or “protective response” refers to an immune response mediated by antibodies against an infectious agent, which is exhibited by a vertebrate (e.g., a human), that prevents or ameliorates an infection or reduces at least one symptom thereof. VLPs of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates SARS-CoV-2 infection or reduces at least one symptom thereof.

“Purified” or “Substantially purified” general refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

As used herein, the term “spike receptor binding domain” (RBD) refers to a part of a virus located on its ‘spike’ domain that allows it to dock to body receptors to gain entry into cells.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein, the term “vaccine” refers to a formulation which contains VLPs of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

A “vector” is capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The vector is usually a plasmid designed for gene expression in cells. The term is used interchangeable with the terms “nucleic acid expression vector”, “expression plasmid”, and “expression cassette”.

As used herein, the terms “virus-like particle”, “VLP”, “recombinant virus-like particle” or “recombinant VLP” refer to a nonreplicating, viral shell. VLPs are generally composed of one or more viral proteins, such as, but not limited to those proteins referred to as capsid, coat, shell, surface and/or envelope proteins, or particle-forming polypeptides derived from these proteins. VLPs can also be described as “enveloped” if they contain a cell derived lipid membrane of the SARS-CoV-2 described here or non-enveloped if assembly with protein without a lipid membrane. The terms nanoparticles or nanospheres have also been used to described virus particles which do not defer in composition from the virus like particles delineated in this disclosure. VLPs can form spontaneously upon recombinant expression of the protein in an appropriate expression system. Methods for producing particular VLPs are known in the art and discussed more fully below. The presence of VLPs following recombinant expression of viral proteins can be detected using conventional techniques known in the art, such as by electron microscopy, biophysical and immunological characterizations, and the like. See, e.g., Baker et al., Biophys. J. (1991) 60:1445-1456; Hagensee et al., J. Virol. (1994) 68:4503-4505. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions. Additional methods of VLP purification include but are not limited to chromatographic techniques such as affinity, ion exchange, size exclusion, and reverse phase procedures.

As used herein, the term “about” for a numerical value means+3% of the numerical value.

In accordance with one or more embodiments, disclosed herein is a SARS-CoV-2 VLP that includes a modified spike (S) glycoprotein of SARS-CoV-2, a matrix (M) protein of SARS-CoV-2, and an envelope (E) protein of SARS-CoV-2. The modified S glycoprotein includes an S1 domain and an S2 domain.

In one or more embodiments, the S1 domain is 400-450 residues, 450-500 residues, 500-550 residues, 550-600 residues, 600-650 residues, 650-700 residues or 700-750 residues. In at least one embodiment, the S1 domain comprises residues 1-675. In one or more embodiments, the S2 domain is 400-450 residues, 450-500 residues, 500-550 residues, 550-600 residues, 600-650 residues, 650-700 residues or 700-750 residues. In at least one embodiment, the S2 domain is comprises residues 677-1400.

The modified S glycoprotein can include at least one of the following modifications: (i) linking the S1 and S2 domains via generation of disulfides bonds between the S1 and S2 domains; (ii) linking intra-polypeptide and inter-polypeptide S2 helices of the S2 domain; and (iii) substitution of one or more non-cysteine residues with a cysteine residue to generate one or more disulfide bonds. The modifications to the S glycoprotein can stabilize a prefusion conformation of the S glycoprotein and/or prohibit a transition to a post-fusion structure.

In at least one embodiment, the linking of the S1 and S2 domains can result from one or more of the following pairs of cysteine substitutions: (i) A653C at the S1 domain and A694C at the S2 domain; (ii) S659C at the S1 domain and S698C at the S2 domain; and (iii) C662C at the S1 domain and M697C at the S2 domain. In one or more embodiments, the linking of intra-polypeptide and inter-polypeptide S2 helices of the S2 domain to one another can result from one or more of the following pairs of cysteine substitutions: (i) Y707C and T883C at the S2 domain; and (ii) V705C and T883C at the S2 domain. In at least one embodiment, the substitution of one or more non-cysteine residues with a cysteine residue generates one or more disulfide bonds that prohibit the spike receptor binding domain (RBD) from a conformational change which includes one or more of the following substitutions: (i) A570C at the S1 domain and V963C at the S2 domain; (ii) D571C at the S1 domain and S967C at the S2 domain; and (iii) K558C at the S1 domain and N282C at the S2 domain.

In one or more embodiments, other modifications to the S glycoproteins can be made. For example, in at least one embodiment, the modifications to the S glycoprotein can include a P862C substitution at the S2 domain and A668C at the S1 domain, where these substitutions result in the locking of the S1 domain of one polypeptide chain to the S2 of another polypeptide chain, resulting in the stabilization of a prefusion conformation of the modified S glycoprotein.

In at least one embodiment, the SARS-CoV-2 VLP can comprise a modification to the S glycoprotein which comprises replacing one or more domains of the SARS-CoV-2 S glycoprotein with analogous portions from one or more other coronaviruses to produce a chimeric or mosaic S glycoprotein. For example, that modification may comprise domain swapping in which, a portion of the S glycoprotein such as the receptor binding domain (RBD) or a complete S1 domain or portion thereof all are exchanged with homologous regions from antigenic variants or another coronavirus of the coronaviridae family. This chimera or hybrid molecules may allow for the design of multivalent, broadly protective, universal or pancoronavirus vaccines. Similarly, for example, the exchange of the transmembrane domain or endodomain of the spike enables contact optimization with homologous or heterologous morphogenesis factor such as the matrix M, the envelope E or nucleocapsid N proteins.

In one or more embodiments, the RBD domain is 100-150 residues, 150-200 residues, 200-250 residues, 250-300 residues, 300-350 residues, or 350-400 residues. In at least one embodiment, the RBD domain comprises residues 319-527.

In one or more embodiments, the domains that are replaced in the S glycoprotein have a size of 100-150 residues, 150-200 residues, 200-250 residues, 250-300 residues, 300-350 residues, 350-400 residues, 400-450 residues, 450-500 residues, 500-550 residues, 550-600 residues, 600-650 residues, 650-700 residues or 700-750 residues.

In at least one embodiment, the modified S glycoprotein is further coexpressed with a nucleocapsid (N) protein of SARS-CoV-2.

In at least one embodiment, the modifications to the S glycoprotein include at least one of the following pairs of cysteine substitutions: (i) A653C at the S1 domain and A694C at the S2 domain; (ii) S659C at the S1 domain and S698C at the S2 domain; (iii) C662C at the S1 domain and M697C at the S2 domain; (iv) V705C and T883C at the S2 domain; (v) A570C at the S1 domain and V963C at the S2 domain; (vi) D571C at the S1 domain and S967C at the S2 domain; (vii) Y707C and T883C at the S2 domain; and (vii) K558C at the S1 domain and N282C at the S2 domain.

In accordance with one or more embodiments, the SARS-CoV-2 VLP described herein is suitable for the preparation of a SARS-CoV-2 vaccine. In at least one embodiment, the VLPs described herein can also be suitable for the preparation of vaccines for one or more other coronaviruses, such as severe acute respiratory syndrome coronavirus (SARS-CoV) or Middle East respiratory syndrome coronavirus (MERS-CoV).

In one or more embodiments, the present application also provides an expression plasmid for the assembly of a VLP of the present application. The expression plasmid comprises genes encoding the coronavirus structural and surface proteins described above.

Also described herein is a method for producing a SARS-CoV-2 VLP, the method comprises introducing into a host cell at least one expression plasmid suitable for the assembly of the SARS-CoV-2 VLP, where the expression plasmid is introduced into the host cell under conditions such that the host cell produces the SARS-CoV-2 VLP. In one or more embodiments, the host cell is a eukaryotic cell, such as a mammalian cell. In at least one embodiment, the eukaryotic cell is stably modified to continuously produce a VLP vaccine, such as a SARS-CoV-2 VLP vaccine.

The present application further provides an immunogenic composition comprising at least one SARS-CoV-2 VLP of the present application. In one or more embodiments, a carrier is optionally present in the compositions described herein. Typically, a carrier is a molecule that does not itself induce the production of antibodies harmful to the individual receiving the composition. Suitable carriers are typically large, slowly metabolized macromolecules such as proteins, polysaccharides, polylactic acids, polyglycollic acids, polymeric amino acids, amino acid copolymers, lipid aggregates (such as oil droplets or liposomes), and inactive virus particles. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J P, et al., J Microencapsul. 14(2):197-210, 1997; O'Hagan D T, et al., Vaccine 11(2):149-54, 1993. Such carriers are well known to those of ordinary skill in the art.

Additionally, these carriers may function as immunostimulating agents (“adjuvants”). Exemplary adjuvants include, but are not limited to: (1) aluminum salts (alum), such as aluminum hydroxide, aluminum phosphate, aluminum sulfate, etc.; (2) oil-in-water emulsion formulations (with or without other specific immuno stimulating agents such as muramyl peptides or bacterial cell wall components), such as for example (a) MF59 (International Publication No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, MT) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detoxu); (3) saponin adjuvants, such as Stimulon™. (Cambridge Bioscience, Worcester, Mass.) may be used or particle generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, etc.), macrophage colony stimulating factor (M-CSF), tumor necrosis factor (TNF), beta chemokines (MIP, 1-alpha, 1-beta Rantes, etc.); (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO 93/13202 and WO 92/19265); and (7) other substances that act as immunostimulating agents to enhance the effectiveness of the composition.

Also described herein is a method of generating an immune response to one or more coronaviruses in a subject. The method comprises administering an effective amount of the immunogenic composition of the present application to a subject. An appropriate effective amount can be determined by one of skill in the art. Such an amount will fall in a relatively broad range that can be determined through routine trials and will generally be an amount on the order of about 0.1 μg to about 10 (or more) mg, more preferably about 1 μg to about 300 μg, of VLP/antigen. In one or more embodiments, the immunogenic composition is administered nasally, mucosally or parenterally. In another aspect of the method, the subject is a human. In another aspect, the immune response vaccinates the subject against one or more coronaviruses. For instance, in at least one embodiment, the immune response vaccinates the subject against SARS-CoV-2.

In one or more embodiments, the immunogenic composition may induce a humoral immune response in the subject administered the immunogenic composition. In some embodiments, the induced humoral immune response may be specific for SARS-CoV-2. The humoral immune response may be induced in the subject administered the immunogenic composition by about 1.5-fold to about 100-fold, about 2-fold to about 90-fold, or about 3-fold to about 80-fold. The humoral immune response can be induced in the subject administered the immunogenic composition by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more. The humoral immune response induced by the immunogenic composition may include an increased level of neutralizing antibodies associated with the subject administered the immunogenic composition as compared to a subject that is not administered the immunogenic composition. The neutralizing antibodies may be specific for SARS-CoV-2. The neutralizing antibodies can provide protection against and/or treatment of SARS-CoV-2 infection and its associated pathologies in the subject administered the immunogenic composition.

In one or more embodiments, the humoral immune response induced by the immunogenic composition may include an increased level of IgG antibodies associated with the subject administered the immunogenic composition as compared to a subject not administered the immunogenic composition.

In at least one embodiment, the humoral response may be cross-reactive against two or more strains of SARS-CoV-2. The level of IgG antibody associated with the subject administered the immunogenic composition may be increased by about 1.5-fold to about 100-fold, about 2-fold to about 50-fold, or about 3-fold to about 25-fold as compared to the subject not administered the immunogenic composition. The level of IgG antibody associated with the subject administered the immunogenic composition can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more.

The VLPs and compositions of the present application can be administered to a subject by any mode of delivery, including, for example, by parenteral injection (e.g. subcutaneously, intraperitoneally, intravenously, intramuscularly, or to the interstitial space of a tissue), or by rectal, oral (e.g. tablet, spray), vaginal, topical, transdermal (e.g. see WO99/27961) or transcutaneous (e.g. see WO02/074244 and WO02/064162), intranasal (e.g. see WO03/028760), ocular, aural, pulmonary or other mucosal administration and/or inhalation of powder compositions. Multiple doses can be administered by the same or different routes. In a preferred embodiment, the doses are intranasally administered.

The VLPs (and VLP-containing compositions) can be administered prior to, concurrent with, or subsequent to delivery of other vaccines. Also, the site of VLP administration may be the same or different as other vaccine compositions that are being administered.

Dosage treatment with the VLP composition may be a single dose schedule or a multiple dose schedule. A multiple dose schedule is one in which a primary course of vaccination may be with 1-10 separate doses, followed by other doses given at subsequent time intervals, chosen to maintain and/or reinforce the immune response, for example at 1-4 months for a second dose, and if needed, a subsequent dose(s) after several months. The dosage regimen will also, at least in part, be determined by the potency of the modality, the vaccine delivery employed, the need of the subject and be dependent on the judgment of the practitioner.

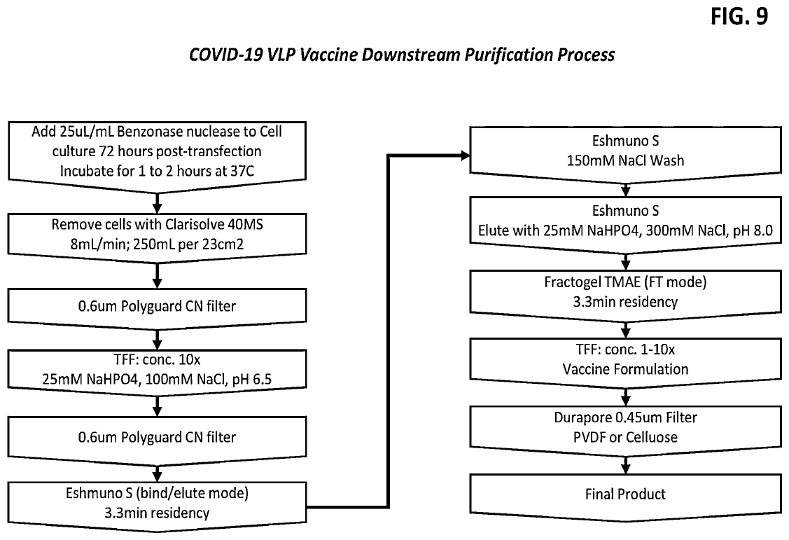

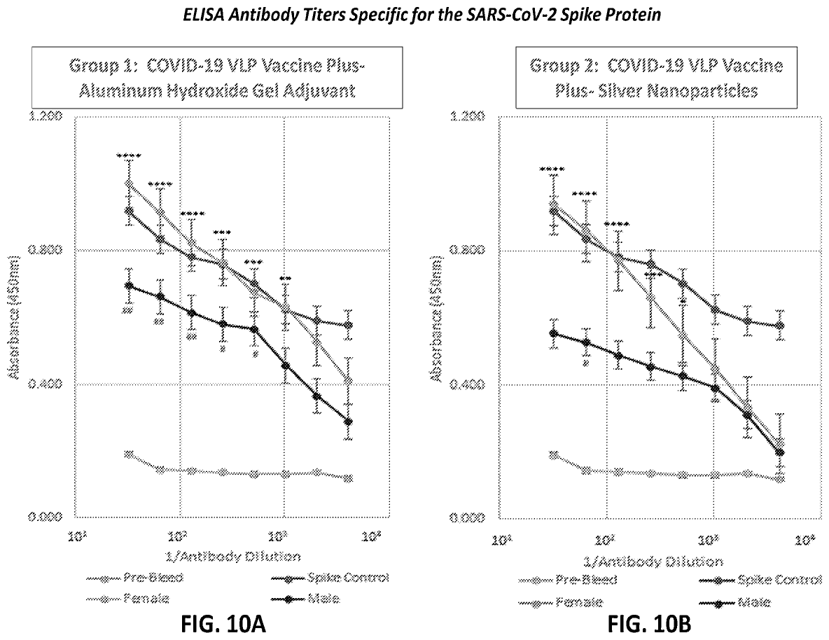

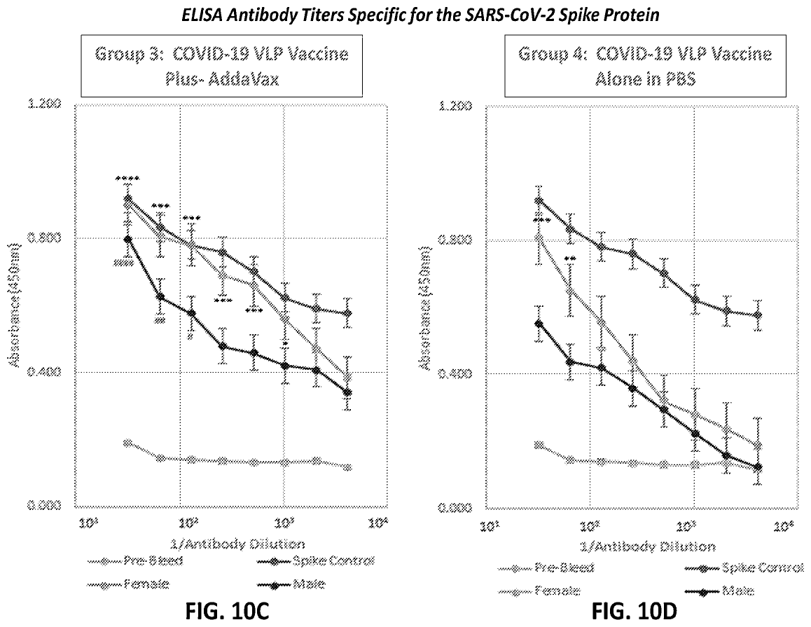

Also described herein is a SARS-CoV-2 VLP vaccine based one or more VLPs of the present application. The SARS-CoV-2 VLP vaccine is described in further detail below with reference to FIGS. 9 and 10A-10D. Also described herein is a purification method for the SARS-CoV-2 VLP vaccine, which is described in further detail below with reference to FIG. 9.

### Examples

Coronavirus virus-like particles (VLP) assembly and production. We have developed a new methodology to produce recombinant virus-like particles (VLPs) utilizing suspension cultures of mammalian cells and state-of-the-art fermentation technology. This strategy allows us to manufacture large quantities of VLPs providing a suitable system for rapid escalation of manufacturing to meet demand. We have constructed a single expression plasmid carrying optimized genes of SARS-CoV-2 structural proteins (S, M, E and N or S, M, E) which is used for the production of coronavirus virus-like particles (COVID-19 VLPs). Specifically, the SARS-CoV-2 structural proteins include the spike (S), envelope (E), membrane (M) and nucleocapsid (N) proteins. The N protein holds the RNA genome, while the other three structural proteins are components of the viral envelope. The S protein (S glycoprotein) allows for the virus to attach to the membrane of a host cell. The S protein comprises an S1 domain that mediates the attachment and an S2 domain that mediates the fusion of the viral cellular membrane to the host cell.

An exemplary amino acid sequence of the S glycoprotein of SARS-CoV-2 is shown below.

Original S glycoprotein (Wuhan strain, MN988669) (SEQ ID NO: 1):

M F V F L V L L P L V S S Q C V N L
T T R T Q L P P A Y T N S F T R G V
Y Y P D K V F R S S V L H S T Q D L
F L P F F S N V T W F H A I H V S G
T N G T K R F D N P V L P F N D G V
Y F A S T E K S N I I R G W I F G T
T L D S K T Q S L L I V N N A T N V
V I K V C E F Q F C N D P F L G V Y
Y H K N N K S W M E S E F R V Y S S
A N N C T F E Y V S Q P F L M D L E
G K Q G N F K N L R E F V F K N I D
G Y F K I Y S K H T P I N L V R D L
P Q G F S A L E P L V D L P I G I N
I T R F Q T L L A L H R S Y L T P G
D S S S G W T A G A A A Y Y V G Y L
Q P R T F L L K Y N E N G T I T D A
V D C A L D P L S E T K C T L K S F
T V E K G I Y Q T S N F R V Q P T E
S I V R F P N I T N L C P F G E V E
N A T R F A S V Y A W N R K R I S N
C V A D Y S V L Y N S A S F S T F K
C Y G V S P T K L N D L C F T N V Y
A D S F V I R G D E V R Q I A P G Q
T G K I A D Y N Y K L P D D F T G C
V I A W N S N N L D S K V G G N Y N
Y L Y R L F R K S N L K P F E R D I
S T E I Y Q A G S T P C N G V E G F
N C Y F P L Q S Y G F Q P T N G V G
Y Q P Y R V V V L S F E L L H A P A
T V C G P K K S T N L V K N K C V N
F N F N G L T G T G V L T E S N K K
F L P F Q Q F G R D I A D T T D A V
R D P Q T L E I L D I T P C S F G G
V S V I T P G T N T S N Q V A V L Y
Q D V N C T E V P V A I H A D Q L T
P T W R V Y S T G S N V F Q T R A G
C L I G A E H V N N S Y E C D I P I
G A G I C A S Y Q T Q T N S P R R A
R S V A S Q S I I A Y T M S L G A E
N S V A Y S N N S I A I P T N F T I
S V T T E I L P V S M T K T S V D C
T M Y I C G D S T E C S N I L L Q Y
G S F C T Q L N R A L T G I A V E Q
D K N T Q E V F A Q V K Q I Y K T P
P I K D F G G F N F S Q I L P D P S
K P S K R S F I E D L L F N K V T L
A D A G F I K Q Y G D C L G D I A A
R D L I C A Q K F N G L T V L P P L
L T D E M I A Q Y T S A L L A G T I
T S G W T F G A G A A L Q I P F A M
Q M A Y R F N G I G V T Q N V L Y E
N Q K L I A N Q F N S A I G K I Q D
S L S S T A S A L G K L Q D V V N Q
N A Q A L N T L V K Q L S S N F G A
I S S V I N D I L S R L D K V E A E
V Q I D R L I T G R L Q S L Q T Y V
T Q Q L I R A A E I R A S A N L A A
T K M S E C V L G Q S K R V D F C G
K G Y H L M S F P Q S A P H G V V F
L H V T Y V P A Q E K N F T T A P A
I C H D G K A H F P R E G V F V S N
G T H W F V T Q R N F Y E P Q I I T
T D N T F V S G N C D V V I G I V N
N T V Y D P L Q P E L D S F K E E L
D K Y F K N H T S P D V D L G D I S
G I N A S V V N I Q K E I D R L N E
V A K N L N E S L I D L Q E L G K Y
E Q Y I K W P W Y I W L G F I A G L
I A I V M V T I M L C C M T S C C S
C L K G C C S C G S C C K F D E D D
S E P V L K G V K L H Y T *

An exemplary amino acid sequence of the M protein of SARS-CoV-2 is shown below.

M from Wuhan strain (MN988669) (SEQ ID NO: 2):

M A D S N G T I T V E E L K K L L E
Q W N L V I G F L F L T W I C L L Q
F A Y A N R N R F L Y I I K L I F L
W L L W P V T L A C F V L A A V Y R
I N W I T G G I A I A M A C L V G L
M W L S Y F I A S F R L F A R T R S
M W S F N P E T N I L L N V P L H G
T I L T R P L L E S E L V I G A V I
L R G H L R I A G H H L G R C D I K
D L P K E I T V A T S R T L S Y Y K
L G A S Q R V A G D S G F A A Y S R
Y R I G N Y K L N T D H S S S S D N
I A L L V Q *

An exemplary amino acid sequence of the E protein of SARS-CoV-2 is shown below.

E from Wuhan strain (MN988669) (SEQ ID NO: 3):

M Y S F V S E E T G T L I V N S V L L F L A F V V F L L V T L A I L T
A L R L C A Y C C N I V N V S L V K P S F Y V Y S R V K N L N S S R V
P D L L V *

An exemplary amino acid sequence of the N protein of SARS-CoV-2 is shown below.

N from Wuhan strain (MN988669) (SEQ ID NO: 4):

M S D N G P Q N Q R N A P R I T F G G P S D S T G S N Q N G E R S G A
R S K Q R R P Q G L P N N T A S W F T A L T Q H G K E D L K F P R G Q
G V P I N T N S S P D D Q I G Y Y R R A T R R I R G G D G K M K D L S
P R W Y F Y Y L G T G P E A G L P Y G A N K D G I I W V A T E G A L N
T P K D H I G T R N P A N N A A I V L Q L P Q G T T L P K G F Y A E G
S R G G S Q A S S R S S S R S R N S S R N S T P G S S R G T S P A R M
A G N G G D A A L A L L L L D R I N Q L E S K M S G K G Q Q Q Q G Q T
V T K K S A A E A S K K P R Q K R T A T K A Y N V T Q A F G R R G P E
Q T Q G N F G D Q E L I R Q G T D Y K H W P Q I A Q F A P S A S A F F
G M S R I G M E V T P S G T W L T Y T G A I K L D D K D P N F K D Q V
I L L N K H I D A Y K T F P P T E P K K D K K K K A D E T Q A L P Q R
Q K K Q Q T V T L L P A A D L D D F S K Q L Q Q S M S S A D S T Q A *

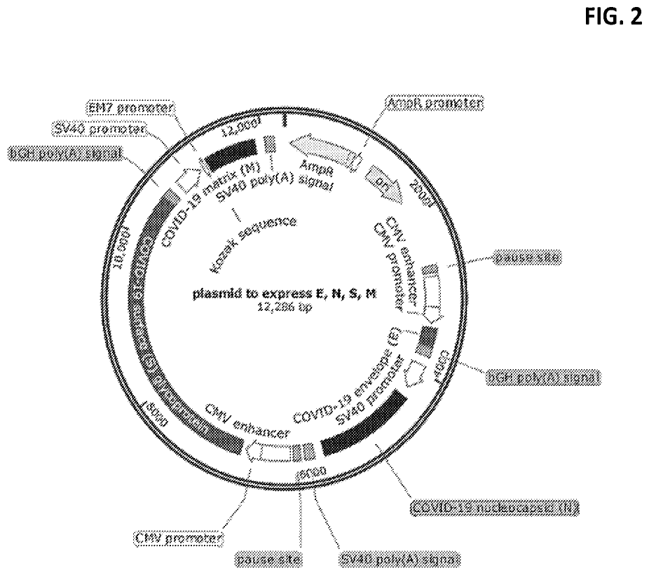

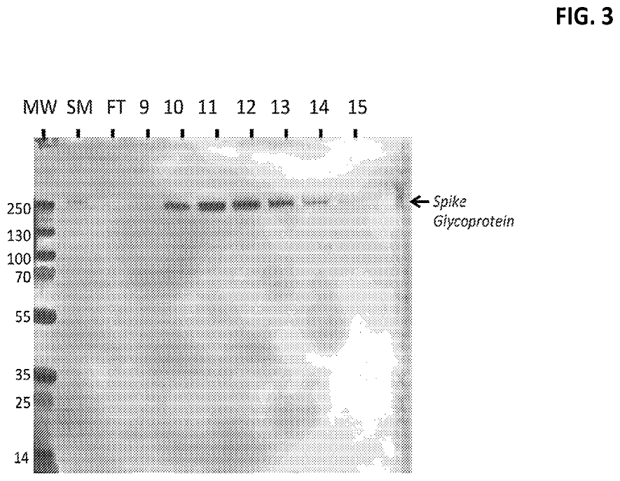

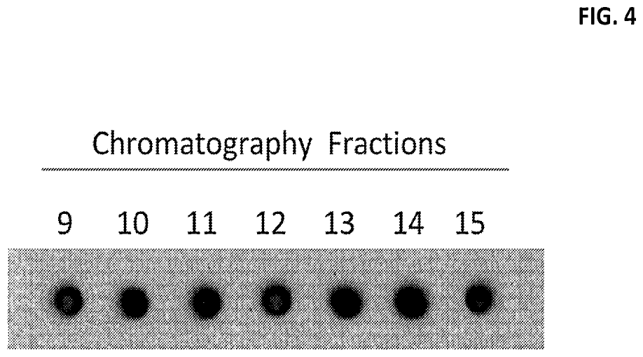

One example of the expression plasmid carrying optimized genes of SARS-CoV-2 structural proteins is shown at FIG. 2. Transfection of a suspension culture of HEK-293 cells grown in chemically defined medium with one of our single plasmids produced coronavirus VLPs that were detected in the culture supernatant. Subsequently, VLP material obtained from the culture supernatant is purified using different downstream processes including successive filtrations (e.g. depth, tangential, etc.), concentration and buffer exchange followed by chromatography purification, sterile filtration and formulation. Western blot of purified material revealed the presence of the spike protein; one example is shown in FIG. 3. Furthermore, dot blot analysis of similar fractions also demonstrated expression of the matrix protein, which comigrated with S and is an essential structural component for particles formation; and example is shown in FIG. 4.

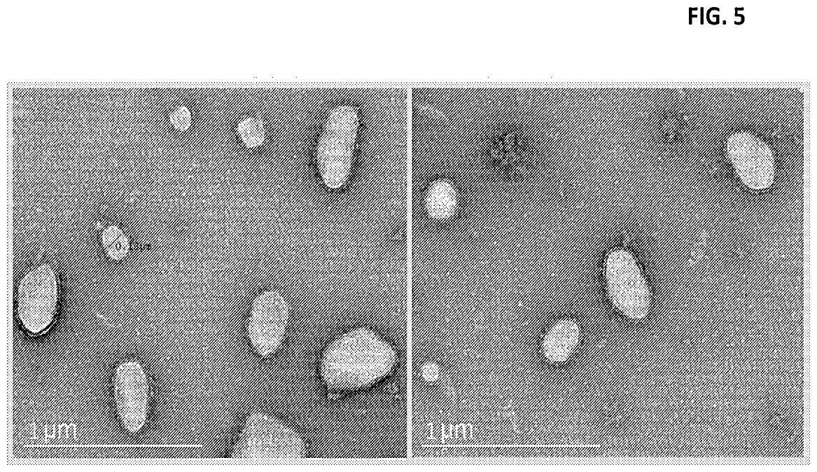

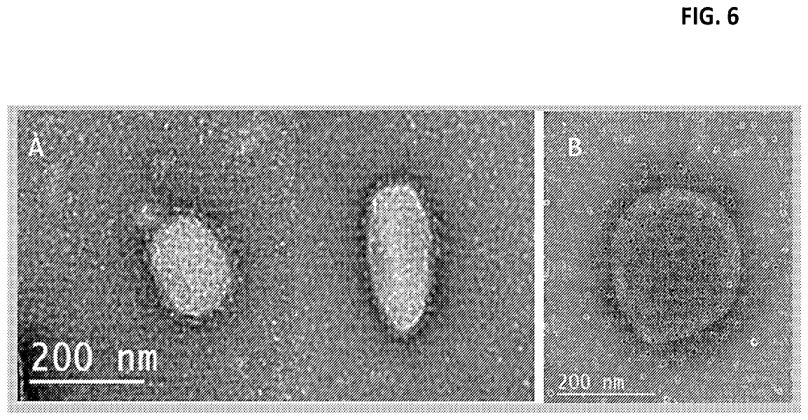

To verify VLP assembly, we examined the purified material by negative staining electron microscopy. This analysis revealed that indeed the VLPs were assembled and exhibit size and morphology similar to the native coronavirus, one example is shown in FIG. 5. Visualization of the typical club shape structure of the spike projecting from the surface of the particles is evident and they resemble those of native coronavirus, FIG. 6A. Furthermore, immunogold labeling electron microscopy using a specific anti-spike antibody demonstrates that club shape structures displayed on the surface of the particles are indeed composed of the S protein, FIG. 6B. Assembly of VLPs with native spike protein may contain a reduced number of spike proteins due to a high degree of instability or shading from the particle. If furin-like proteases cut the molecule at the cleavage sites, S protein stability may be affected due to the lack of disulfide bridges between the S1 and S2 domains leaving only other forces (H-bonds, hydrophobic interactions, etc.) which could be of insufficient strength to hold these domains together. This reasoning provides further rationale for the modification of S in order to enhance its stability and as a result its immunogenic properties.

The described technology allows for the production of VLP particles assembled with the most important structural and immunogenic components of the SARS-CoV-2, and they resemble native virion in morphology, size and biochemical composition as shown. Furthermore, modified version of the VLP building block are used (for example mutated S, M or E) in order to ensure stability of the spike, improve immunogenic properties, enhance VLP production and structural stability, etc. These modifications enhance not only VLP production and stability, but also their immunogenic properties which is critical for making safe and effective coronavirus vaccines. The multi-antigenic composition, the diverse origin of components, and the display of distinct and conformational modified surface spike proteins provide a unique and versatile approach to formulate a vaccine for protecting against SARS-CoV-2 as well as, based on its composition, to other coronavirus as monovalent, multivalent, universal or pancoronavirus vaccines.

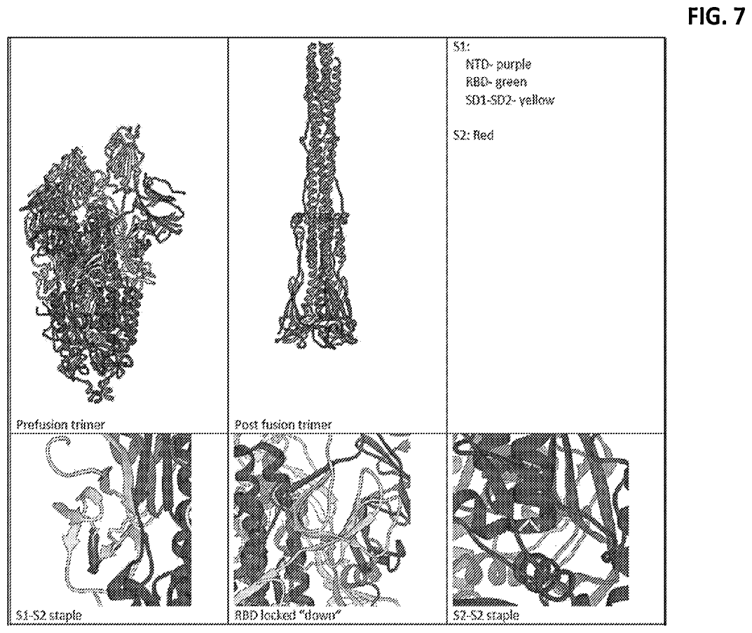

VLP components modification. The SARS-CoV-2 glycoprotein spike (S) is a metastable trimeric molecule displayed on the surface of the virion in a prefusion conformation which is less stable and transforms into a more stable post-fusion state that mediates membrane fusion. These conformational changes result from significant rearrangement of structural motifs within the domains of the molecule as shown in FIG. 7. It is anticipated that this structural rearrangement of S alters the antigenic epitopes composition of the prefusion molecule, which may exhibit the most relevant determinants for the induction of potent neutralizing antibodies. As it has been shown, the S1 domain of the prefusion conformation reveals an extended up-position that is suitable for receptor binding whereas the down-position resulting from the conformational change does not. These conformational variations should have a significant impact on the immunogenic properties of the S molecule. Thus, in order to stabilize the molecule and preserve potentially potent neutralizing sites, we introduced amino acid changes to create disulfide bridges, which will lock the molecule in its prefusion conformation.

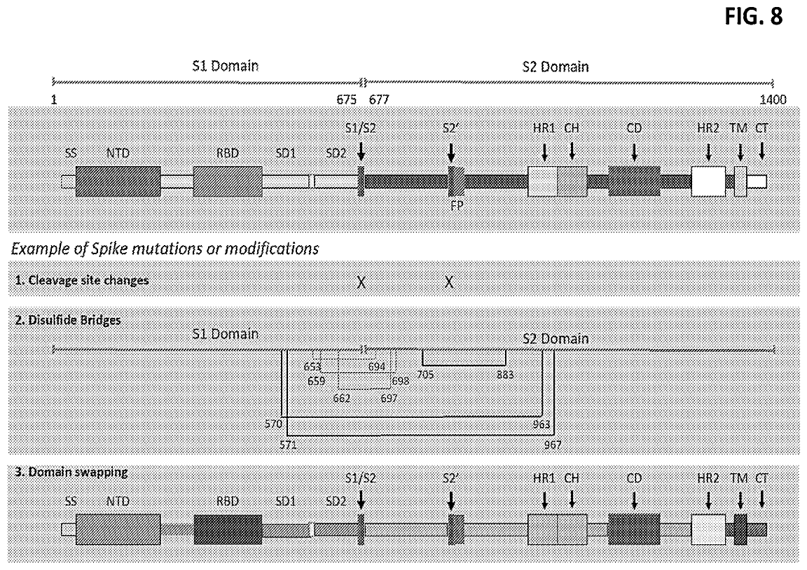

We have used a structure-based approach to identify a number of neighboring amino acids that can be substituted for cysteine (Cys, or C) to generate disulfide bonds that would further stabilize the prefusion and prohibit the transition to the post-fusion structure. This strategy involves stapling the S1 and S2 domains to one another via disulfide bonds such that the S1 cannot peel away from the S2, (FIG. 7 lower panel and FIG. 8) as has been hypothesized, to allow the S2 to undergo the structural conformations necessary to drive membrane fusion. A second set of substitutions are aimed to staple intra- and inter-polypeptide neighboring S2 helices of the S2 domain to one another such that the spike trimer cannot undergo the large conformational changes observed in the post-fusion structure (FIG. 7 lower panel and FIG. 8). Finally, we have identified amino acids whose substitution to Cys will generate disulfide bonds that will prohibit the spike receptor binding domain (RBD) from experiencing the “up” and “down” conformation that are necessary for receptor binding (FIG. 7 lower panel and FIG. 8) The substitution sites, the neighboring amino acid sequence, and strategy are identified in the table below and some illustrated in FIG. 8.

| Amino acid | | |
|---|---|---|
| substitution | Sequence | Strategy |
| A653C | (SEQ. ID NO: 15) LIGAEHV | Staple S1 to S2 |
| A694C | (SEQ. ID NO: 16) SIIAYTM | |
| S659C | (SEQ. ID NO: 17) VNNSYEC | Staple S1 to S2 |
| S698C | (SEQ. ID NO: 18) YTMSLGA | |
| C662C | (SEQ. ID NO: 19) SYECDIP | Staple S1 to S2 |
| M697C | (SEQ. ID NO. 20) AYTMSLG | |
| A570C | (SEQ. ID NO: 21) RDIADTT | Lock RBD “down” |
| V963C | (SEQ. ID NO: 22) NTLVKQL | |
| D571C | (SEQ. ID NO: 23) DIADTTD | Lock RBD “down” |
| S967C | (SEQ. ID NO: 24) KQLSSNF | |
| K558C | (SEQ. ID NO: 25) SNKKFLP | Lock RBD “down” |
| N282C | (SEQ. ID NO: 26) YNENGTI | |
| P862C | (SEQ. ID NO: 27) TVLPPLL | Lock S1 of one chain to S2 of |
| A668C | (SEQ. ID NO: 28) PIGAGIC | another chain. This P862 was an |
| | | engineered substitution to stabilize |
| | | prefusion conformation. |
| Y707C | (SEQ. ID NO: 29) SVAYSNN | Lock S2 of one chain to S2 of |
| T883C | (SEQ. ID NO: 30) GTITSGW | another chain. Prevents postfusion |
| | | from occurring |
| V705C | (SEQ. ID NO: 31) ENSVAYS | Lock S2 of one chain to S2 of |
| T883C | (SEQ. ID NO: 30) GTITSGW | another chain. Prevents postfusion |
| | | from occurring |

In one or more embodiments, one or more of the above mutations can be used for the VLPs of the present application. Furthermore, amino acid changes in the S1/S2 protease cleavage site (FIG. 8) will preclude cleavage keeping the domain together which will further enhance molecular stability. The incorporation of a stable S on the surface of the VLP will display relevant epitopes for the elicitation of potent neutralizing antibodies and therefore afford protection against infection with SARS-CoV-2.

Additional examples of modification of the spike protein includes the formation of hetero-trimeric molecules (wild type spike is a homo-trimer formed with three identical monomers), which may be assembled with three distinct antigenic monomeric variants such that the hetero-trimer displays antigenic sites specific of each one monomer, providing greater antigenic diversity and therefore broader vaccine coverage, e.g. monomers derived from the Delta, Gamma, and Beta SARS-CoV-2 variants. To attain formation of hetero-trimers, the molecular contacts among the monomers interface are modified in such a way that associations are only allowed among the three distinct monomers directing the sole assembly of hetero-trimers while precluding alternative arrangements. In accordance with one or more embodiments, all these changes on the spike protein can be implemented in molecules with or without the proline modifications previously described by others.

Further modifications of the spike (S) glycoprotein involve swapping one or more domains of the molecule with analogous portions from other coronaviruses producing chimeric or mosaic spikes that when used as VLP vaccine may broaden protection against infection with multiple coronaviruses. Similarly, changes in other structural components of the VLP such as the matrix protein M, the envelope protein E, or the nucleocapsid N, may also affect particle formation and production, stability, immunological properties, etc.

### Description of SARS-CoV-2 VLP Production

In accordance with one or more embodiments, the SARS-CoV-2 virus-like particles (VLPs) are produced by transfection of suspension culture of mammalian cells with a plasmid that expresses the four structural proteins of SARS-CoV-2 virus (e.g. spike surface protein, membrane protein M, the nucleocapsid N, and the envelope protein E). Sixteen hours post-transfection, cell division is control by the addition of valproic acid, which enhances protein production and VLP yield. The culture is continued from 72 hours at which point the vaccine material in purified using a downstream process that is outlined in FIG. 9.

Description of SARS-CoV-2 VLP purification (downstream process—FIG. 9)

FIG. 9 displays a flow chart outlining the purification stages of the SARS-CoV-2 VLP vaccine following production in suspension culture of mammalian cells in accordance with one or more embodiments. The process produces a highly purified final vaccine product. With reference now to FIG. 9, approximately 72 hours post-transfection, the purification of the vaccine material is initiated. First, the transfected cell culture is incubated with a nuclease, e.g. Benzonase in order to degrade the DNA present in the culture. In one or more embodiments, the cell culture can be incubated for 1-2 hours at a temperature of 37° C. Subsequently, cells are removed and culture clarified via filtration through a filtration device, such as a Clarisolve 40 mS filter (MilliporeSigma). This filtration can be run at different flow rates per squared centimeter of the Clarisolve 40 ms media (e.g. 8 mL/min; 250 ml per cm2). The clarified culture is then immediately pass through a second filtration step (e.g., 0.6 um Polyguard CN filter). Filtered material is then concentrated (e.g., 10× concentration) and buffer exchanged via tangential flow filtration (TFF) (e.g., using 25 mM NaHPO₄, 100 mM NaCl, pH 6.5). Following TFF, the VLP material is once again filtered through a 0.6 um device and subsequently loaded onto a cation-exchange chromatography resin (e.g. Eshmuno S, MilliporeSigma; 3.3 min residency). The retained vaccine material is eluted from the chromatography column utilizing a step salt gradient (e.g., 150 mM NaCl wash; elute with 25 mM NaHPO₄, 300 mM NaCl, pH 8.0) and those fractions containing the vaccine product further purified by directly passing it through an anion-exchange chromatography resin (e.g. Fractogel TMAE HiCap, MilliporeSigma; 3.3 min residency). Subsequently, the vaccine product is concentrated (e.g., 1-10× concentration) via tangential flow filtration and buffer exchanged into its final formulation, which is then passed through and sterilizing filters (e.g., a Durapore PVDF 0.45 um or cellulose) and ready for the fill and finish stage.

Immunization of small animal models with the SARS-CoV-2 VLP resulted in the elicitation of a strong immune response which is required for protection against SARS-CoV-2 infection.

Examination of antibody production and its magnitude was evaluated by the ELISA (enzyme-linked immunosorbent assay) using as antigen a purified recombinant spike protein derived from the Wuhan coronavirus. This assay showed that the VLP vaccine formulated with different adjuvant elicited a robust anti-spike specific antibody immune response (see FIGS. 10A-10D).

Specifically, FIGS. 10A-10D provide graphs that display the results of ELISA antibody titers specific for the SARS-CoV-2 spike protein in accordance with one or more embodiments. In this example, mice were immunized twice using a prime and booster regimen with COVID-19 VLP (SARS-CoV-2 VLP) vaccine formulated with three different adjuvants (groups 1-3) or PBS control (group 4). Serum samples were collected three weeks after the booster dose and specific antibodies titers against the Wuhan spike protein measured via ELISA. The VLP vaccine was prepared with the South African variant of the SARS-CoV-2 virus (identified as Beta [B] with the new WHO nomenclature) while the spike protein antigen used to coat the ELISA plate was derived from the Wuhan as it also was the serum control.

The COVID-19 VLP (SARS-CoV-2 VLP) vaccine elicited the production of significant levels of anti-Spike antibody titers as compared to the pre-immunization control serum (gray dots-pre-immunization bleed). Statistical significance was determined by a two-way ANOVA with a Tukey post hoc test. Asterisks represent significance between Female (Covid VLP Vaccine) and Mouse Serum from the Pre-Immunization Bleed control. The antigenic differences between the spike displayed in the VLP vaccine, South African variant (Beta) and the Wuhan spike used in the assay may explain the slightly difference in antibody titers between the vaccine and the positive control serum, which was also produced with the Wuhan spike. However, all adjuvanted VLP vaccine formulations elicited statistically significant higher antibody titers as compared to those of the pre-immunization bleed serum samples control. Thus, the VLP vaccine formulations are highly immunogenic and the technology suitable for multivalent vaccine compositions.

Amino acid sequences and nucleotide sequences for proteins of various SAR-CoV-2 strains with regards to the VLPs described herein, in accordance with one or more embodiments, including modified sequences, are shown below. Modifications from wild type (if present) in the various sequences are shown in underline.

### Amino Acid Sequences

S glycoprotein with Triple mutations and S1-S2 mutation (Wuhan strain, MN988669)(SEQ ID NO: 5):

M F V F L V L L P L V S S Q C V N L T T R T Q L P P A Y T N S F T R G
V Y Y P D K V F R S S V L H S T Q D L F L P F F S N V T W F H A I H V
S G T N G T K R F D N P V L P F N D G V Y F A S T E K S N I I R G W I
F G T T L D S K T Q S L L I V N N A T N V V I K V C E F Q F C N D P F
L G V Y Y H K N N K S W M E S E F R V Y S S A N N C T F E Y V S Q P F
L M D L E G K Q G N F K N L R E F V F K N I D G Y F K I Y S K H T P I
N L V R D L P Q G F S A L E P L V D L P I G I N I T R F Q T L L A L H
R S Y L T P G D S S S G W T A G A A A Y Y V G Y L Q P R T F L L K Y N
E N G T I T D A V D C A L D P L S E T K C T L K S F T V E K G I Y Q T
S N F R V Q P T E S I V R F P N I T N L C P F G E V E N A T R F A S V
Y A W N R K R I S N C V A D Y S V L Y N S A S F S T F K C Y G V S P T
K L N D L C F T N V Y A D S F V I R G D E V R Q I A P G Q T G K I A D
Y N Y K L P D D F T G C V I A W N S N N L D S K V G G N Y N Y L Y R L
F R K S N L K P F E R D I S T E I Y Q A G S T P C N G V E G F N C Y F
P L Q S Y G F Q P T N G V G Y Q P Y R V V V L S F E L L H A P A T V C
G P K K S T N L V K N K C V N F N F N G L T G T G V L T E S N K K F L
P F Q Q F G R D I A D T T D A V R D P Q T L E I L D I T P C S F G G V
S V I T P G T N T S N Q V A V L Y Q D V N C T E V P V A I H A D Q L T
P T W R V Y S T G S N V F Q T R A G C L I G C E H V N N C Y E C D I P
I G A G I C A S Y Q T Q T N S P S G S A S V A S Q S I I C Y T C C L G
A E N S V A Y S N N S I A I P T N F T I S V T T E I L P V S M T K T S
V D C T M Y I C G D S T E C S N I L L Q Y G S F C T Q L N R A L T G I
A V E Q D K N T Q E V F A Q V K Q T Y K T P P I K D F G G F N F S Q I
L P D P S K P S K R S F I E D L L F N K V T L A D A G F I K Q Y G D C
L G D I A A R D L I C A Q K F N G L T V L P P L L T D E M I A Q Y T S
A L L A G T I T S G W T F G A G A A L Q I P F A M Q M A Y R F N G I G
V T Q N V L Y E N Q K L I A N Q F N S A I G K I Q D S L S S T A S A L
G K L Q D V V N Q N A Q A L N T L V K Q L S S N F G A I S S V L N D I
L S R L D K V E A E V Q I D R L I T G R L Q S L Q T Y V T Q Q L I R A
A E I R A S A N L A A T K M S E C V L G Q S K R V D F C G K G Y H L M
S F P Q S A P H G V V F L H V T Y V P A Q E K N F T T A P A I C H D G
K A H F P R E G V F V S N G T H W F V T Q R N F Y E P Q I I T T D N T
F V S G N C D V V I G I V N N T V Y D P L Q P E L D S F K E E L D K Y
F K N H T S P D V D L G D I S G I N A S V V N I Q K E I D R L N E V A
K N L N E S L I D L Q E L G K Y E Q Y I K W P W Y I W L G F I A G L I
A I V M V T I M L C C M T S C C S C L K G C C S C G S C C K F D E D D
S E P V L K G V K L H Y T *

S glycoprotein from South Africa strain with mutations (B.1.351) (SEQ ID NO: 6):

M F V F L V L L P L V S S Q C V N L T T R T Q L P P A Y T N S F T R G
V Y Y P D K V F R S S V L H S T Q D L F L P F F S N V T W F H A I H V
S G T N G T K R F A N P V L P F N D G V Y F A S T E K S N I I R G W I
F G T T L D S K T Q S L L I V N N A T N V V I K V C E F Q F C N D P F
L G V Y Y H K N N K S W M E S E F R V Y S S A N N C T F E Y V S Q P F
L M D L E G K Q G N F K N L R E F V F K N I D G Y F K I Y S K H T P I
N L V R G L P Q G F S A L E P L V D L P I G I N I T R F Q T L H R S Y
L T P G D S S S G W T A G A A A Y Y V G Y L Q P R T F L L K Y N E N G
T I T D A V D C A L D P L S E T K C T L K S F T V E K G I Y Q T S N F
R V Q P T E S I V R F P N I T N L C P F G E V E N A T R F A S V Y A W
N R K R I S N C V A D Y S V L Y N S A S F S T F K C Y G V S P T K L N
D L C F T N V Y A D S F V I R G D E V R Q I A P G Q T G N I A D Y N Y
K L P D D F T G C V I A W N S N N L D S K V G G N Y N Y L Y R L F R K
S N L K P F E R D I S T E I Y Q A G S T P C N G V K G F N C Y F P L Q
S Y G F Q P T Y G V G Y Q P Y R V V V L S F E L L H A P A T V C G P K
K S T N L V K N K C V N F N F N G L T G T G V L T E S N K K F L P F Q
Q F G R D I A D T T D A V R D P Q T L E I L D I T P C S F G G V S V I
T P G T N T S N Q V A V L Y Q G V N C T E V P V A I H A D Q L T P T W
R V Y S T G S N V F Q T R A G C L I G A E H V N N S Y E C D I P I G A
G I C A S Y Q T Q T N S P S G S A S V A S Q S I I A Y T M S L G V E N
S V A Y S N N S I A I P T N F T I S V T T E I L P V S M T K T S V D C
T M Y I C G D S T E C S N L L L Q Y G S F C T Q L N R A L T G I A V E
Q D K N T Q E V F A Q V K Q T Y K T P P I K D F G G F N F S Q I L P D
P S K P S K R S F I E D L L F N K V T L A D A G F I K Q Y G D C L G D
I A A R D L I C A Q K E N G L T V L P P L L T D E M I A Q Y T S A L L
A G T I T S G W T F G A G A A L Q I P F A M Q M A Y R E N G I G V T Q
N V L Y E N Q K L I A N Q F N S A I G K I Q D S L S S T A S A L G K L
Q D V V N Q N A Q A L N T L V K Q L S S N F G A I S S V I N D I L S R
L D P P E A E V Q I D R L I T G R L Q S L Q T Y V T Q Q L I R A A E I
R A S A N L A A T K M S E C V L G Q S K R V D F C G K G Y H L M S F P
Q S A P H G V V F L H V T Y V P A Q E K N F T T A P A I C H D G K A H
F P R E G V F V S N G T H W F V T Q R N F Y E P Q I I T T D N T F V S
G N C D V V I G I V N N T V Y D P L Q P E L D S F K E E L D K Y F K N
H T S P D V D L G D I S G I N A S V V N I Q K E I D R L N E V A K N L
N E S L I D L Q E L G K Y E Q Y I K W P W Y I W L G F I A G L I A I V
M V T I M L C C M T S C C S C L K G C C S C G S C C K F D E D D S E P
V L K G V K L H Y T *

S glycoprotein from India strain with mutations ((B.1.617) (SEQ ID NO: 7):

M F V F L V L L P L V S S Q C V N L T T R T Q L P P A Y T N S F T R G
V Y Y P D K V F R S S V L H S T Q D L F L P F F S N V T W F H A I H V
S G T N G T K R F D N P V L P F N D G V Y F A S I E K S N I I R G W I
F G T T L D S K T Q S L L I V N N A T N V V I K V C E F Q F C N D P F
L D V Y Y H K N N K S W M K S E F R V Y S S A N N C T F E Y V S Q P F
L M D L E G K Q G N F K N L R E F V F K N I D G Y F K I Y S K H T P I
N L V R D L P Q G F S A L E P L V D L P I G I N I T R F Q T L L A L H
R S Y L T P G D S S S G W T A G A A A Y Y V G Y L Q P R T F L L K Y N
E N G T I T D A V D C A L D P L S E T K C T L K S F T V E K G I Y Q T
S N F R V Q P T E S I V R F P N I T N L C P F G E V E N A T R F A S V
Y A W N R K R I S N C V A D Y S V L Y N S A S F S T F K C Y G V S P T
K L N D L C F T N V Y A D S F V I R G D E V R Q I A P G Q T G K I A D
Y N Y K L P D D F T G C V I A W N S N N L D S K V G G N Y N Y R Y R L
F R K S N L K P F E R D I S T E I Y Q A G S T P C N G V Q G F N C Y F
P L Q S Y G F Q P T N G V G Y Q P Y R V V V L S F E L L H A P A T V C
G P K K S T N L V K N K C V N F N F N G L T G T G V L T E S N K K F L
P F Q Q F G R D I A D T T D A V R D P Q T L E I L D I T P C S F G G V
S V I T P G T N T S N Q V A V L Y Q G V N C T E V P V A I H A D Q L T
P T W R V Y S T G S N V F Q T R A G C L I G A E H V N N S Y E C D I P
I G A G I C A S Y Q T Q T N S R S G S A S V A S Q S I I A Y T M S L G
A E N S V A Y S N N S I A I P T N F T I S V T T E I L P V S M T K T S
V D C T M Y I C G D S T E C S N I L L Q Y G S F C T Q L N R A L T G I
A V E Q D K N T Q E V F A Q V K Q I Y K T P P I K D F G G F N F S Q I
L P D P S K P S K R S F I E D L L F N K V T L A D A G F I K Q Y G D C
L G D I A A R D L I C A Q K F N G L T V L P P L L T D E M I A Q Y T S
A L L A G T I T S G W T F G A G A A L Q I P F A M Q M A Y R F N G I G
V T Q N V L Y E N Q K L I A N Q F N S A I G K I Q D S L S S T A S A L
G K L Q D V V N Q N A Q A L N T L V K Q L S S N F G A I S S V I N D I
L S R L D P P E A E V Q I D R L I T G R L Q S L Q T Y V T Q Q L I R A
A E I R A S A N L A A T K M S E C V L G Q S K R V D F C G K G Y H L M
S F P Q S A P H G V V F L H V T Y V P A H E K N F T T A P A I C H D G
K A H F P R E G V F V S N G T H W F V T Q R N F Y E P Q I I T T D N T
F V S G N C D V V I G I V N N T V Y D P L Q P E L D S F K E E L D K Y
F K N H T S P D V D L G D I S G I N A S V V N I Q K E I D R L N E V A
K N L N E S L I D L Q E L G K Y E Q Y I K W P W Y I W L G F I A G L I
A I V M V T I M L C C M T S C C S C L K G C C S C G S C C K F D E D D
S E P V L K G V K L H Y T *

### Nucleotide Sequences

Original S glycoprotein (Wuhan strain, MN988669) (SEQ ID NO: 8):

ATGTTCGTGT TCCTCGTGCT GCTGCCCCTG GTGTCTAGCC AGTGCGTGAA CCTGACCACC
AGAACCCAGC TGCCTCCAGC CTACACCAAC AGCTTCACCA GGGGAGTGTA CTACCCCGAC
AAGGTGTTCC GGAGCAGCGT GCTGCATAGC ACCCAGGACC TGTTCCTGCC CTTCTTCAGC
AACGTGACTT GGTTCCACGC CATCCACGTC AGCGGAACCA ACGGCACCAA GCGCTTTGAC
AACCCAGTGC TGCCCTTCAA CGACGGAGTG TACTTCGCCA GCACCGAGAA GAGCAACATC
ATCCGGGGTT GGATCTTCGG CACCACACTG GACAGCAAGA CCCAGAGCCT GCTGATCGTG
AACAACGCCA CCAACGTCGT GATCAAGGTC TGCGAGTTCC AGTTTTGCAA CGACCCCTTC
CTGGGCGTGT ACTACCACAA GAACAACAAG TCTTGGATGG AGAGCGAGTT CCGGGTGTAC
AGCAGCGCCA ACAATTGCAC CTTCGAGTAC GTGTCCCAGC CCTTCCTGAT GGACCTGGAA
GGCAAGCAGG GCAACTTCAA GAACCTGCGG GAGTTCGTGT TCAAGAACAT CGACGGCTAC
TTCAAGATCT ACAGCAAGCA CACCCCCATC AACCTCGTGA GAGACCTGCC ACAGGGCTTC
AGCGCTCTGG AACCTCTGGT GGATCTGCCA ATCGGCATCA ACATCACCCG GTTCCAGACC
CTGCTGGCTC TGCACAGAAG CTACCTGACC CCAGGCGATT CTTCTAGCGG CTGGACAGCA
GGAGCCGCCG CTTATTACGT GGGCTATCTG CAGCCCCGGA CCTTCCTGCT GAAGTACAAC
GAGAACGGCA CCATCACCGA CGCAGTGGAT TGCGCTCTGG ATCCTCTGAG CGAGACCAAG
TGCACCCTGA AGAGCTTCAC CGTGGAGAAG GGCATCTACC AGACCAGCAA CTTCCGGGTG
CAGCCTACAG AGAGCATCGT GCGGTTCCCT AACATCACCA ACCTCTGCCC CTTCGGCGAG
GTGTTCAACG CCACCAGATT CGCCAGCGTC TACGCCTGGA ACCGGAAGCG GATCAGCAAT
TGCGTGGCCG ACTACAGCGT GCTGTACAAC AGCGCCAGCT TCAGCACCTT CAAGTGCTAC
GGCGTGTCCC CTACCAAGCT GAACGATCTG TGCTTCACCA ACGTGTACGC CGACAGCTTC
GTGATCAGGG GCGACGAAGT GCGACAGATC GCCCCAGGAC AGACAGGAAA GATCGCCGAT
TACAACTACA AGCTGCCCGA CGACTTCACC GGCTGCGTGA TCGCTTGGAA CAGCAACAAC
CTGGACAGCA AGGTCGGCGG CAACTACAAC TACCTGTACC GGCTGTTCCG GAAGAGCAAC
CTGAAGCCCT TCGAGCGGGA CATCAGCACA GAGATCTACC AGGCCGGCAG CACACCTTGT
AACGGCGTGG AGGGCTTCAA CTGCTACTTC CCTCTGCAGA GCTACGGCTT CCAGCCTACA
AACGGCGTGG GATACCAGCC TTACAGGGTG GTGGTGCTGA GCTTCGAACT GCTGCACGCT
CCAGCCACCG TCTGCGGACC TAAAAAGAGC ACCAACCTGG TGAAGAACAA GTGCGTGAAT
TTCAACTTCA ACGGCCTGAC CGGAACCGGC GTGCTGACCG AGAGCAACAA GAAGTTCCTG
CCCTTCCAGC AGTTCGGCAG AGACATCGCA GACACCACAG ACGCCGTGAG AGATCCTCAG
ACCCTGGAGA TCCTGGACAT CACCCCTTGC AGCTTTGGCG GAGTGTCCGT GATCACCCCA
GGAACCAACA CCAGCAATCA GGTGGCCGTG CTGTACCAGG ACGTGAATTG CACCGAGGTG
CCAGTGGCCA TTCACGCAGA TCAGCTGACC CCTACCTGGA GAGTGTACAG CACCGGAAGC
AACGTGTTCC AGACCAGGGC AGGTTGCCTG ATCGGAGCCG AGCACGTGAA CAACAGCTAC
GAGTGCGACA TCCCCATCGG CGCCGGCATT TGCGCCTCTT ACCAGACCCA GACCAACAGC
CCTAGAAGAG CCAGAAGCGT GGCCTCTCAG AGCATCATCG CCTACACCAT GAGCCTGGGA
GCCGAGAACA GCGTGGCCTA CAGCAACAAC AGCATCGCCA TCCCCACCAA CTTCACCATC
AGCGTGACCA CCGAGATTCT GCCCGTGTCT ATGACCAAGA CCAGCGTCGA CTGCACCATG
TACATCTGCG GCGACAGCAC CGAGTGCTCT AACCTGCTGC TGCAGTACGG CTCCTTTTGC
ACCCAGCTGA ACAGGGCTCT GACAGGCATC GCAGTGGAGC AGGACAAGAA CACCCAGGAG
GTCTTCGCCC AGGTCAAGCA GATCTACAAG ACCCCCCCTA TCAAGGACTT CGGCGGCTTT
AACTTCAGCC AGATCCTGCC CGATCCCAGC AAGCCTAGCA AGCGGAGCTT CATCGAGGAC
CTGCTGTTCA ACAAGGTCAC CCTGGCCGAC GCAGGCTTCA TCAAGCAGTA CGGCGATTGC
CTGGGCGACA TTGCCGCCAG AGACCTGATT TGCGCCCAGA AGTTCAACGG CCTGACAGTG
CTGCCTCCTC TGCTGACCGA CGAGATGATC GCCCAGTATA CAAGCGCTCT GCTGGCAGGA
ACCATCACCA GCGGTTGGAC CTTTGGAGCC GGAGCCGCTC TGCAGATCCC TTTTGCCATG
CAGATGGCCT ACCGCTTCAA CGGAATCGGC GTGACCCAGA ACGTGCTCTA CGAGAACCAG
AAGCTGATCG CCAACCAGTT CAACAGCGCC ATCGGCAAGA TCCAGGACAG CCTGAGCAGC
ACAGCTAGCG CTCTGGGCAA GCTGCAGGAT GTGGTGAACC AGAACGCCCA GGCTCTGAAC
ACCCTGGTGA AGCAGCTGAG CAGCAACTTC GGAGCCATCA GCAGCGTGCT GAACGACATC
CTGAGCAGGC TGGACAAGGT GGAAGCCGAA GTGCAGATCG ACAGGCTGAT CACCGGCAGA
CTGCAGTCTC TGCAGACCTA CGTGACCCAG CAGCTGATCA GAGCCGCCGA GATCAGAGCT
AGCGCTAATC TGGCCGCCAC CAAGATGAGC GAGTGCGTGC TGGGACAGAG CAAGAGAGTG
GACTTCTGCG GCAAGGGCTA CCACCTGATG AGCTTTCCTC AGAGCGCTCC TCACGGAGTG
GTGTTTCTGC ACGTGACCTA CGTGCCAGCC CAGGAGAAGA ACTTCACCAC AGCCCCAGCC
ATTTGCCACG ACGGAAAGGC CCACTTCCCC AGAGAAGGCG TGTTCGTGTC CAACGGCACC
CATTGGTTCG TGACCCAGCG GAACTTCTAC GAGCCCCAGA TCATCACCAC CGACAACACC
TTCGTGAGCG GCAATTGCGA CGTGGTCATC GGCATCGTGA ACAACACCGT GTACGACCCC
CTGCAGCCAG AGCTGGACTC CTTCAAGGAG GAGCTGGACA AGTACTTCAA GAACCACACC
AGCCCAGACG TGGACCTGGG AGACATCAGC GGCATCAACG CCAGCGTGGT GAACATCCAG
AAGGAGATCG ACAGGCTGAA CGAGGTGGCC AAGAACCTGA ACGAGAGCCT GATCGACCTG
CAGGAGCTGG GCAAGTACGA GCAGTACATC AAGTGGCCTT GGTACATTTG GCTGGGCTTC
ATCGCCGGCC TGATCGCAAT CGTCATGGTG ACCATCATGC TCTGTTGCAT GACCAGTTGC
TGCAGCTGCC TGAAGGGTTG CTGCAGTTGC GGGAGTTGCT GCAAGTTCGA CGAGGACGAC
AGCGAGCCAG TGCTGAAAGG CGTGAAGCTG CACTACACCT AA

M protein from Wuhan strain (MN988669) (SEQ ID NO: 9):

ATGGCCGACT CTAACGGCAC CATCACCGTG GAGGAGCTGA AGAAGCTGCT CGAGCAGTGG
AACCTGGTCA TCGGCTTCCT GTTCCTCACC TGGATTTGCC TGCTGCAGTT CGCCTACGCC
AACCGGAACC GGTTCCTGTA CATCATCAAG CTGATCTTCC TCTGGCTCCT CTGGCCCGTG
ACACTGGCTT GTTTCGTGCT GGCAGCCGTG TACCGGATCA ATTGGATCAC CGGCGGCATC
GCTATCGCTA TGGCTTGTCT CGTGGGCCTG ATGTGGCTGA GCTACTTCAT CGCCAGCTTC
CGGCTGTTCG CCAGAACCAG AAGCATGTGG AGCTTCAACC CCGAGACCAA CATCCTGCTG
AACGTGCCTC TGCACGGCAC AATCCTGACC AGACCTCTGC TGGAGAGCGA GCTGGTCATC
GGAGCAGTGA TCCTGAGAGG CCACCTGAGA ATCGCCGGAC ATCATCTGGG ACGGTGCGAC
ATCAAGGACC TGCCCAAGGA GATCACAGTG GCCACAAGCC GGACCCTGAG CTACTACAAG
CTGGGAGCCA GCCAGAGAGT GGCAGGAGAT AGCGGATTCG CCGCCTACAG CAGATACAGG
ATCGGCAACT ACAAGCTGAA CACCGACCAC AGCAGCAGCA GCGATAACAT CGCCCTGCTG
GTGCAGTAA

E protein from Wuhan strain (MN988669) (SEQ ID NO: 10):

ATGTACAGCT TCGTGTCCGA GGAGACCGGA ACCCTGATCG TGAACTCCGT GCTGCTGTTC
CTGGCCTTTG TGGTGTTCCT GCTGGTGACC CTGGCCATTC TGACAGCTCT CAGGCTCTGC
GCCTATTGCT GCAACATCGT GAACGTGTCC CTGGTGAAGC CCAGCTTCTA CGTGTACAGC
CGCGTGAAGA ACCTGAACAG CAGCAGGGTG CCAGATCTGC TGGTGTAA

N protein from Wuhan strain (MN988669) (SEQ ID NO: 11):

ATGAGCGACA ACGGACCTCA GAACCAGCGG AACGCCCCTA GAATCACCTT TGGCGGCCCT
AGCGATAGCA CCGGCAGCAA TCAGAACGGC GAGAGAAGCG GCGCCAGATC TAAGCAGAGG
AGACCTCAGG GCCTGCCTAA CAATACCGCC TCTTGGTTCA CCGCTCTGAC ACAGCACGGC
AAGGAGGACC TGAAGTTCCC TAGGGGCCAG GGCGTGCCTA TCAATACCAA CAGCAGCCCC
GACGATCAGA TCGGCTACTA CAGGAGGGCC ACCAGAAGGA TCAGAGGCGG AGACGGCAAG
ATGAAGGACC TGAGCCCCCG CTGGTACTTT TACTATCTGG GCACAGGACC AGAGGCCGGA
CTGCCTTACG GAGCCAACAA GGACGGCATC ATTTGGGTGG CCACCGAAGG AGCCCTGAAC
ACCCCTAAGG ACCACATCGG CACCAGGAAC CCAGCCAATA ACGCCGCTAT CGTGCTGCAG
CTGCCTCAGG GAACAACACT GCCTAAGGGC TTTTACGCCG AGGGCAGCAG AGGAGGAAGC
CAGGCCTCTA GCAGAAGCAG CAGCAGAAGC AGGAACAGCA GCAGAAACAG CACCCCAGGC
AGCAGCAGAG GAACCAGCCC AGCCAGAATG GCAGGAAACG GAGGAGACGC AGCTCTGGCT
CTGCTGCTGC TGGATAGGCT GAACCAGCTG GAGAGCAAGA TGAGCGGCAA GGGACAGCAG
CAGCAGGGAC AGACCGTGAC CAAGAAGAGC GCCGCCGAAG CCTCTAAGAA GCCTAGACAG
AAGCGGACCG CCACCAAGGC TTACAACGTG ACCCAGGCCT TCGGCAGAAG AGGACCAGAA
CAGACCCAGG GCAACTTCGG CGATCAGGAA CTGATCCGGC AGGGCACCGA CTACAAGCAT
TGGCCTCAGA TCGCCCAGTT TGCTCCTAGC GCCAGCGCCT TTTTCGGCAT GAGCAGGATC
GGCATGGAAG TGACCCCTAG CGGAACTTGG CTGACCTACA CCGGAGCCAT CAAGCTGGAC
GACAAGGACC CCAACTTCAA GGACCAGGTC ATCCTGCTGA ACAAGCACAT CGACGCCTAC
AAGACCTTCC CTCCCACCGA GCCCAAGAAG GACAAGAAGA AGAAGGCCGA CGAGACCCAG
GCTCTGCCTC AGAGGCAGAA GAAGCAGCAG ACCGTGACAC TGCTGCCAGC AGCAGATCTG
GACGACTTCA GCAAGCAGCT GCAGCAGAGC ATGAGCAGCG CCGATTCTAC ACAGGCCTAA

S glycoprotein with Triple mutations and S1-S2 mutation (Wuhan strain, MN988669) (SEQ ID NO: 12):

ATGTTCGTGT TCCTCGTGCT GCTGCCCCTG GTGTCTAGCC AGTGCGTGAA CCTGACCACC
AGAACCCAGC TGCCTCCAGC CTACACCAAC AGCTTCACCA GGGGAGTGTA CTACCCCGAC
AAGGTGTTCC GGAGCAGCGT GCTGCATAGC ACCCAGGACC TGTTCCTGCC CTTCTTCAGC
AACGTGACTT GGTTCCACGC CATCCACGTC AGCGGAACCA ACGGCACCAA GCGCTTTGAC
AACCCAGTGC TGCCCTTCAA CGACGGAGTG TACTTCGCCA GCACCGAGAA GAGCAACATC
ATCCGGGGTT GGATCTTCGG CACCACACTG GACAGCAAGA CCCAGAGCCT GCTGATCGTG
AACAACGCCA CCAACGTCGT GATCAAGGTC TGCGAGTTCC AGTTTTGCAA CGACCCCTTC
CTGGGCGTGT ACTACCACAA GAACAACAAG TCTTGGATGG AGAGCGAGTT CCGGGTGTAC
AGCAGCGCCA ACAATTGCAC CTTCGAGTAC GTGTCCCAGC CCTTCCTGAT GGACCTGGAA
GGCAAGCAGG GCAACTTCAA GAACCTGCGG GAGTTCGTGT TCAAGAACAT CGACGGCTAC
TTCAAGATCT ACAGCAAGCA CACCCCCATC AACCTCGTGA GAGACCTGCC ACAGGGCTTC
AGCGCTCTGG AACCTCTGGT GGATCTGCCA ATCGGCATCA ACATCACCCG GTTCCAGACC
CTGCTGGCTC TGCACAGAAG CTACCTGACC CCAGGCGATT CTTCTAGCGG CTGGACAGCA
GGAGCCGCCG CTTATTACGT GGGCTATCTG CAGCCCCGGA CCTTCCTGCT GAAGTACAAC
GAGAACGGCA CCATCACCGA CGCAGTGGAT TGCGCTCTGG ATCCTCTGAG CGAGACCAAG
TGCACCCTGA AGAGCTTCAC CGTGGAGAAG GGCATCTACC AGACCAGCAA CTTCCGGGTG
CAGCCTACAG AGAGCATCGT GCGGTTCCCT AACATCACCA ACCTCTGCCC CTTCGGCGAG
GTGTTCAACG CCACCAGATT CGCCAGCGTC TACGCCTGGA ACCGGAAGCG GATCAGCAAT
TGCGTGGCCG ACTACAGCGT GCTGTACAAC AGCGCCAGCT TCAGCACCTT CAAGTGCTAC
GGCGTGTCCC CTACCAAGCT GAACGATCTG TGCTTCACCA ACGTGTACGC CGACAGCTTC
GTGATCAGGG GCGACGAAGT GCGACAGATC GCCCCAGGAC AGACAGGAAA GATCGCCGAT
TACAACTACA AGCTGCCCGA CGACTTCACC GGCTGCGTGA TCGCTTGGAA CAGCAACAAC
CTGGACAGCA AGGTCGGCGG CAACTACAAC TACCTGTACC GGCTGTTCCG GAAGAGCAAC
CTGAAGCCCT TCGAGCGGGA CATCAGCACA GAGATCTACC AGGCCGGCAG CACACCTTGT
AACGGCGTGG AGGGCTTCAA CTGCTACTTC CCTCTGCAGA GCTACGGCTT CCAGCCTACA
AACGGCGTGG GATACCAGCC TTACAGGGTG GTGGTGCTGA GCTTCGAACT GCTGCACGCT
CCAGCCACCG TCTGCGGACC TAAAAAGAGC ACCAACCTGG TGAAGAACAA GTGCGTGAAT
TTCAACTTCA ACGGCCTGAC CGGAACCGGC GTGCTGACCG AGAGCAACAA GAAGTTCCTG
CCCTTCCAGC AGTTCGGCAG AGACATCGCA GACACCACAG ACGCCGTGAG AGATCCTCAG
ACCCTGGAGA TCCTGGACAT CACCCCTTGC AGCTTTGGCG GAGTGTCCGT GATCACCCCA
GGAACCAACA CCAGCAATCA GGTGGCCGTG CTGTACCAGG ACGTGAATTG CACCGAGGTG
CCAGTGGCCA TTCACGCAGA TCAGCTGACC CCTACCTGGA GAGTGTACAG CACCGGAAGC
AACGTGTTCC AGACCAGGGC AGGTTGCCTG ATCGGATGCG AGCACGTGAA CAACTGCTAC
GAGTGCGACA TCCCCATCGG CGCCGGCATT TGCGCCTCTT ACCAGACCCA GACCAACAGC
CCTAGTGGTA GCGCTAGCGT GGCCTCTCAG AGCATCATCT GCTACACCTG CTGCCTGGGA
GCCGAGAACA GCGTGGCCTA CAGCAACAAC AGCATCGCCA TCCCCACCAA CTTCACCATC
AGCGTGACCA CCGAGATTCT GCCCGTGTCT ATGACCAAGA CCAGCGTCGA CTGCACCATG
TACATCTGCG GCGACAGCAC CGAGTGCTCT AACCTGCTGC TGCAGTACGG CTCCTTTTGC
ACCCAGCTGA ACAGGGCTCT GACAGGCATC GCAGTGGAGC AGGACAAGAA CACCCAGGAG
GTCTTCGCCC AGGTCAAGCA GATCTACAAG ACCCCCCCTA TCAAGGACTT CGGCGGCTTT
AACTTCAGCC AGATCCTGCC CGATCCCAGC AAGCCTAGCA AGCGGAGCTT CATCGAGGAC
CTGCTGTTCA ACAAGGTCAC CCTGGCCGAC GCAGGCTTCA TCAAGCAGTA CGGCGATTGC
CTGGGCGACA TTGCCGCCAG AGACCTGATT TGCGCCCAGA AGTTCAACGG CCTGACAGTG
CTGCCTCCTC TGCTGACCGA CGAGATGATC GCCCAGTATA CAAGCGCTCT GCTGGCAGGA
ACCATCACCA GCGGTTGGAC CTTTGGAGCC GGAGCCGCTC TGCAGATCCC TTTTGCCATG
CAGATGGCCT ACCGCTTCAA CGGAATCGGC GTGACCCAGA ACGTGCTCTA CGAGAACCAG
AAGCTGATCG CCAACCAGTT CAACAGCGCC ATCGGCAAGA TCCAGGACAG CCTGAGCAGC
ACAGCTAGCG CTCTGGGCAA GCTGCAGGAT GTGGTGAACC AGAACGCCCA GGCTCTGAAC
ACCCTGGTGA AGCAGCTGAG CAGCAACTTC GGAGCCATCA GCAGCGTGCT GAACGACATC
CTGAGCAGGC TGGACAAGGT GGAAGCCGAA GTGCAGATCG ACAGGCTGAT CACCGGCAGA
CTGCAGTCTC TGCAGACCTA CGTGACCCAG CAGCTGATCA GAGCCGCCGA GATCAGAGCT
AGCGCTAATC TGGCCGCCAC CAAGATGAGC GAGTGCGTGC TGGGACAGAG CAAGAGAGTG
GACTTCTGCG GCAAGGGCTA CCACCTGATG AGCTTTCCTC AGAGCGCTCC TCACGGAGTG
GTGTTTCTGC ACGTGACCTA CGTGCCAGCC CAGGAGAAGA ACTTCACCAC AGCCCCAGCC
ATTTGCCACG ACGGAAAGGC CCACTTCCCC AGAGAAGGCG TGTTCGTGTC CAACGGCACC
CATTGGTTCG TGACCCAGCG GAACTTCTAC GAGCCCCAGA TCATCACCAC CGACAACACC
TTCGTGAGCG GCAATTGCGA CGTGGTCATC GGCATCGTGA ACAACACCGT GTACGACCCC
CTGCAGCCAG AGCTGGACTC CTTCAAGGAG GAGCTGGACA AGTACTTCAA GAACCACACC
AGCCCAGACG TGGACCTGGG AGACATCAGC GGCATCAACG CCAGCGTGGT GAACATCCAG
AAGGAGATCG ACAGGCTGAA CGAGGTGGCC AAGAACCTGA ACGAGAGCCT GATCGACCTG
CAGGAGCTGG GCAAGTACGA GCAGTACATC AAGTGGCCTT GGTACATTTG GCTGGGCTTC
ATCGCCGGCC TGATCGCAAT CGTCATGGTG ACCATCATGC TCTGTTGCAT GACCAGTTGC
TGCAGCTGCC TGAAGGGTTG CTGCAGTTGC GGGAGTTGCT GCAAGTTCGA CGAGGACGAC
AGCGAGCCAG TGCTGAAAGG CGTGAAGCTG CACTACACCT AA

S glycoprotein from South Africa strain with mutations (B.1.351) (SEQ ID NO: 13):

ATGTTCGTGT TCCTCGTGCT GCTGCCCCTG GTGTCTAGCC AGTGCGTGAA CCTGACCACC
AGAACCCAGC TGCCTCCAGC CTACACCAAC AGCTTCACCA GGGGAGTGTA CTACCCCGAC
AAGGTGTTCC GGAGCAGCGT GCTGCATAGC ACCCAGGACC TGTTCCTGCC CTTCTTCAGC
AACGTGACTT GGTTCCACGC CATCCACGTG TCCGGAACCA ACGGCACCAA GAGATTCGCC
AACCCAGTGC TGCCCTTCAA CGACGGAGTG TACTTCGCCA GCACCGAGAA GAGCAACATC
ATCCGGGGTT GGATCTTCGG CACCACACTG GACAGCAAGA CCCAGAGCCT GCTGATCGTG
AACAACGCCA CCAACGTCGT GATCAAGGTC TGCGAGTTCC AGTTTTGCAA CGACCCCTTC
CTGGGCGTGT ACTACCACAA GAACAACAAG TCTTGGATGG AGAGCGAGTT CCGGGTGTAC
AGCAGCGCCA ACAATTGCAC CTTCGAGTAC GTGTCCCAGC CCTTCCTGAT GGACCTGGAA
GGCAAGCAGG GCAACTTCAA GAACCTGCGG GAGTTCGTGT TCAAGAACAT CGACGGCTAC
TTCAAGATCT ACAGCAAGCA CACCCCCATC AACCTCGTGA GAGGCCTGCC TCAGGGATTC
TCAGCCCTGG AACCTCTGGT GGATCTGCCA ATCGGCATCA ACATCACCCG GTTCCAGACC
CTGCACAGAA GCTACCTGAC CCCAGGCGAT TCTTCTAGCG GCTGGACAGC AGGAGCCGCA
GCTTACTACG TGGGATACCT GCAGCCCAGG ACCTTCCTGC TGAAGTACAA CGAGAACGGC
ACCATCACCG ACGCAGTGGA TTGCGCTCTG GACCCTCTGA GCGAGACCAA GTGTACCCTC
AAGAGCTTCA CCGTGGAGAA GGGCATCTAC CAGACCAGCA ACTTCCGGGT GCAGCCTACA
GAGAGCATCG TGCGGTTCCC CAACATCACC AACCTCTGCC CCTTCGGCGA GGTGTTCAAC
GCTACCAGAT TCGCCAGCGT GTACGCTTGG AACCGGAAGC GGATCAGCAA TTGCGTGGCC
GACTACAGCG TGCTGTACAA CAGCGCCAGC TTCAGCACCT TCAAGTGCTA CGGCGTGTCT
CCCACCAAGC TGAACGACCT CTGCTTCACC AACGTGTACG CCGACAGCTT CGTGATCCGG
GGAGACGAAG TGCGCCAGAT TGCTCCAGGA CAGACCGGCA ACATCGCCGA CTACAACTAC
AAGCTGCCCG ACGACTTCAC CGGCTGCGTG ATCGCCTGGA ACAGCAACAA CCTGGACAGC
AAGGTCGGCG GCAACTACAA CTACCTGTAC CGGCTGTTCC GGAAGAGCAA CCTGAAGCCT
TTCGAGCGGG ACATCAGCAC CGAGATCTAC CAGGCAGGCA GCACACCTTG TAACGGAGTG
AAGGGCTTCA ACTGCTACTT CCCCCTGCAG AGCTACGGCT TTCAGCCTAC CTACGGCGTG
GGATATCAGC CCTACAGGGT GGTGGTGCTG AGCTTTGAGC TGCTGCACGC CCCAGCTACA
GTGTGCGGAC CCAAGAAGAG CACCAACCTG GTGAAGAACA AGTGCGTGAA CTTCAACTTC
AATGGCCTGA CCGGCACAGG AGTGCTGACA GAGAGCAACA AGAAGTTCCT GCCCTTCCAG
CAGTTCGGCA GGGATATCGC CGATACCACC GACGCCGTGA GAGATCCTCA GACACTGGAG
ATCCTGGACA TCACCCCTTG CAGCTTCGGA GGAGTGTCCG TGATCACCCC AGGCACAAAC
ACCAGCAACC AGGTGGCAGT GCTGTACCAG GGCGTGAATT GCACCGAAGT GCCAGTGGCT
ATCCACGCAG ACCAGCTGAC CCCTACTTGG AGGGTGTACA GCACCGGCAG CAACGTGTTC
CAGACCAGAG CAGGTTGTCT GATCGGAGCC GAGCACGTGA ACAACAGCTA CGAGTGCGAC
ATCCCTATCG GAGCCGGCAT TTGCGCCTCT TACCAGACCC AGACCAACTC TCCTAGCGGC
AGCGCCAGCG TGGCCTCTCA GTCTATCATC GCCTACACCA TGAGCCTGGG CGTGGAAAAC
AGCGTGGCCT ACAGCAACAA CAGCATCGCC ATCCCCACCA ACTTCACCAT CAGCGTGACC
ACCGAGATCC TGCCAGTGTC CATGACCAAG ACCAGCGTCG ACTGCACCAT GTACATTTGC
GGCGATAGCA CCGAGTGCTC CAATCTGCTG CTGCAGTACG GCTCCTTTTG CACCCAGCTG
AACAGAGCCC TGACAGGAAT CGCCGTGGAG CAGGACAAGA ACACCCAGGA GGTCTTCGCC
CAGGTCAAGC AGATCTACAA GACCCCCCCC ATCAAGGACT TCGGCGGCTT CAACTTCAGC
CAGATCCTGC CAGATCCCAG CAAGCCTAGC AAGCGGAGCT TCATCGAGGA CCTGCTGTTC
AACAAGGTCA CACTGGCCGA CGCCGGCTTT ATCAAGCAGT ACGGCGATTG CCTGGGCGAC
ATTGCAGCCA GGGACCTGAT TTGCGCCCAG AAGTTCAACG GCCTGACCGT GCTGCCTCCT
CTGCTGACCG ACGAGATGAT CGCCCAGTAC ACAAGCGCCC TGCTGGCCGG AACAATCACA
AGCGGATGGA CATTTGGAGC CGGAGCAGCT CTGCAGATCC CTTTCGCCAT GCAGATGGCC
TACCGCTTCA ACGGAATCGG CGTGACCCAG AACGTGCTCT ACGAGAACCA GAAGCTGATC
GCCAACCAGT TCAACAGCGC CATCGGCAAG ATCCAGGACA GCCTGTCTAG CACAGCCAGC
GCTCTGGGAA AGCTGCAGGA CGTGGTGAAC CAGAACGCTC AGGCCCTGAA TACCCTGGTG
AAGCAGCTGA GCAGCAACTT CGGAGCCATC AGCAGCGTGC TGAACGACAT CCTGAGCAGG
CTGGACCCTC CAGAAGCCGA AGTGCAGATC GACAGGCTGA TCACCGGCAG ACTGCAGTCT
CTGCAGACCT ACGTGACCCA GCAGCTGATC AGAGCCGCAG AGATCAGAGC CAGCGCCAAT
CTGGCAGCTA CCAAGATGAG CGAGTGCGTC CTGGGACAGA GCAAGCGGGT GGACTTTTGC
GGCAAGGGCT ACCACCTGAT GAGCTTCCCT CAGAGCGCTC CTCACGGAGT GGTGTTTCTG
CACGTGACCT ACGTGCCAGC CCAGGAAAAG AACTTCACCA CCGCCCCAGC CATTTGCCAC
GACGGAAAGG CCCACTTTCC TAGAGAGGGC GTGTTCGTGT CCAACGGCAC CCATTGGTTC
GTGACCCAGC GGAACTTCTA CGAGCCCCAG ATCATCACCA CCGACAACAC CTTCGTGAGC
GGCAATTGCG ACGTGGTCAT CGGCATCGTG AACAACACCG TGTACGACCC CCTGCAGCCA
GAGCTGGACT CCTTCAAGGA GGAGCTGGAC AAGTACTTCA AGAACCACAC CAGCCCAGAC
GTGGACCTGG GCGATATCAG CGGCATCAAC GCCAGCGTGG TGAACATCCA GAAGGAGATC
GACAGGCTGA ACGAGGTGGC CAAGAACCTG AACGAGAGCC TGATCGACCT GCAGGAGCTG
GGCAAGTACG AGCAGTACAT CAAGTGGCCT TGGTACATTT GGCTGGGCTT CATCGCCGGC
CTGATCGCCA TCGTCATGGT CACCATCATG CTCTGCTGCA TGACCAGTTG CTGCTCTTGC
CTGAAGGGTT GCTGCAGCTG CGGGTCTTGT TGCAAGTTCG ACGAGGACGA CAGCGAGCCA
GTGCTGAAGG GCGTGAAGCT GCACTACACC TAA

S glycoprotein from India strain with mutations ((B.1.617) (SEQ ID NO: 14):

ATGTTCGTGT TCCTCGTGCT GCTGCCCCTC GTTAGTTCTC AATGCGTGAA CCTGACCACG
CGGACCCAGC TGCCACCCGC CTATACCAAC TCATTTACTC GAGGGGTGTA CTATCCCGAT
AAGGTGTTTC GATCATCTGT ACTCCACAGC ACCCAAGATC TGTTTCTCCC ATTCTTCAGT
AACGTGACCT GGTTTCACGC CATTCACGTG AGTGGCACTA ACGGCACAAA ACGCTTCGAC
AATCCGGTGC TGCCTTTCAA CGACGGCGTA TACTTCGCAT CAATAGAGAA AAGCAATATC
ATCCGGGGTT GGATATTCGG CACAACCCTG GATAGCAAAA CACAAAGTCT GCTCATCGTC
AACAATGCGA CGAACGTAGT GATCAAGGTG TGCGAGTTCC AGTTCTGCAA CGACCCCTTC
CTGGACGTGT ACTATCATAA GAATAATAAG TCCTGGATGA AGTCAGAATT TCGGGTGTAC
TCAAGCGCTA ACAACTGTAC CTTCGAGTAC GTGTCACAAC CATTCCTCAT GGATCTCGAG
GGCAAGCAAG GAAACTTTAA GAACTTGAGA GAGTTCGTTT TCAAGAACAT CGACGGCTAC
TTCAAGATTT ACAGTAAACA TACACCAATC AACCTGGTTA GAGACCTGCC ACAAGGCTTC
TCTGCCCTCG AGCCCCTCGT CGACCTGCCC ATCGGGATCA ATATTACGCG TTTCCAGACA
CTCCTGGCCC TGCACAGGTC ATACCTCACA CCCGGCGACT CATCATCCGG GTGGACTGCA
GGAGCCGCTG CCTACTACGT AGGATACCTG CAGCCCCGCA CCTTCCTGCT GAAGTACAAC
GAGAACGGCA CTATAACCGA CGCCGTGGAT TGCGCTCTCG ATCCCCTTTC TGAGACGAAA
TGCACTCTCA AGTCATTTAC CGTCGAGAAG GGCATTTACC AGACCAGCAA TTTCAGGGTT
CAGCCTACTG AGTCAATCGT GCGATTCCCA AACATCACGA ATCTGTGTCC GTTCGGGGAA
GTCTTCAATG CGACTCGGTT CGCCAGCGTG TACGCCTGGA ATAGAAAACG TATTAGTAAT
TGCGTGGCAG ACTACTCCGT GOTTTACAAC AGCGCTAGTT TCTCTACATT CAAATGCTAC
GGGGTAAGTC CCACAAAGTT GAACGACTTG TGTTTCACAA ACGTATACGC GGACAGCTTC
GTGATCCGTG GCGACGAGGT GCGCCAGATT GCCCCCGGTC AGACAGGGAA AATAGCCGAC
TACAACTACA AGCTTCCCGA CGACTTCACG GGTTGTGTGA TTGCCTGGAA CTCAAATAAC
CTCGACAGCA AAGTCGGTGG GAACTACAAC TATAGGTACA GGCTGTTCCG CAAATCAAAC
CTGAAGCCCT TCGAACGCGA CATCAGTACC GAGATATACC AAGCTGGATC TACTCCATGC
AACGGCGTCC AGGGCTTCAA CTGCTATTTC CCCCTGCAGA GTTACGGGTT TCAGCCTACA
AACGGAGTGG GATATCAGCC TTATCGGGTG GTCGTCCTGA GTTTCGAGCT GCTTCACGCT
CCTGCGACCG TCTGCGGCCC CAAGAAATCA ACGAACCTTG TAAAGAATAA GTGCGTTAAC
TTTAATTTTA ACGGCCTGAC CGGGACTGGG GTGTTGACAG AATCCAATAA GAAATTCCTT
CCATTTCAGC AGTTCGGTAG GGATATAGCA GATACCACCG ACGCCGTGAG GGACCCTCAA
ACGCTCGAAA TCCTCGATAT CACCCCCTGC TCATTCGGCG GGGTTTCCGT CATCACCCCC
GGGACCAACA CCTCAAATCA AGTGGCCGTG CTGTACCAAG GGGTCAATTG TACTGAGGTT
CCGGTGGCAA TCCACGCCGA CCAGCTGACC CCAACCTGGA GAGTGTACTC CACTGGCAGT
AACGTCTTCC AGACCCGCGC TGGTTGCCTC ATCGGCGCCG AGCACGTGAA TAATTCCTAC
GAATGCGATA TCCCTATCGG AGCCGGGATC TGTGCATCAT ACCAAACGCA AACCAACAGC
AGAAGCGGCA GTGCATCCGT GGCATCTCAG AGCATAATCG CGTATACAAT GAGTTTGGGC
GCCGAGAACT CTGTCGCATA TTCCAACAAT TCCATCGCGA TCCCTACGAA CTTCACCATC
TCTGTCACAA CCGAGATACT CCCTGTATCA ATGACTAAAA CTAGTGTGGA CTGCACCATG
TATATCTGCG GCGACAGCAC CGAGTGTTCT AACCTGCTGC TGCAGTACGG AAGCTTCTGC
ACTCAGTTGA ATCGAGCCCT GACGGGCATT GCCGTGGAGC AGGATAAGAA TACGCAGGAA
GTCTTCGCTC AGGTGAAGCA GATCTATAAG ACTCCCCCTA TCAAGGACTT CGGCGGCTTC
AACTTCTCTC AGATTTTGCC CGACCCTTCC AAGCCTTCAA AACGCAGCTT CATCGAGGAC
CTGTTGTTTA ATAAGGTTAC CCTGGCCGAC GCCGGATTTA TTAAGCAGTA CGGGGACTGT
CTCGGGGACA TCGCAGCAAG GGATCTGATA TGCGCCCAGA AATTCAATGG GTTGACAGTA
CTTCCCCCGC TGCTTACTGA CGAGATGATC GCCCAGTATA CGAGCGCGCT CCTGGCCGGG
ACGATTACAT CAGGGTGGAC TTTCGGCGCT GGGGCAGCCC TGCAGATTCC CTTCGCCATG
CAGATGGCCT ACCGCTTCAA CGGCATCGGC GTGACCCAAA ACGTGCTGTA CGAAAATCAG
AAGCTGATCG CAAATCAGTT CAACTCCGCC ATAGGGAAGA TCCAGGATAG CTTGAGCTCA
ACCGCCTCTG CTCTCGGGAA GCTCCAGGAC GTCGTGAATC AGAACGCTCA GGCCCTCAAT
ACCCTGGTCA AGCAGCTCAG TAGCAACTTC GGCGCTATCT CTTCTGTGTT GAACGACATT
CTGAGCAGGC TGGATCCACC GGAAGCAGAG GTCCAGATAG ACCGACTGAT TACTGGAAGG
CTGCAGAGCC TTCAAACTTA CGTAACCCAG CAGCTTATCA GGGCAGCCGA GATAAGGGCC
AGCGCCAACT TGGCCGCGAC GAAGATGTCT GAATGCGTTT TGGGCCAGTC TAAGAGGGTG
GACTTCTGCG GGAAAGGGTA CCACTTGATG TCATTTCCCC AAAGCGCCCC ACACGGAGTG
GTGTTTCTTC ACGTCACCTA CGTGCCCGCT CACGAGAAGA ATTTTACCAC AGCGCCCGCT
ATATGCCACG ACGGTAAGGC CCATTTCCCC AGAGAGGGCG TGTTCGTGAG CAACGGGACT
CATTGGTTCG TCACGCAGCG CAACTTCTAC GAGCCCCAGA TTATCACCAC TGATAATACT
TTCGTCAGCG GCAATTGCGA CGTGGTTATT GGCATCGTAA ATAATACTGT GTACGACCCG
CTGCAGCCCG AGCTCGATTC CTTTAAAGAA GAACTCGACA AGTACTTCAA GAACCACACT
AGCCCCGACG TCGACCTCGG AGATATTTCC GGGATAAACG CCAGCGTCGT GAATATACAG
AAGGAGATCG ATAGACTGAA CGAAGTCGCT AAGAACCTGA ACGAGAGCCT TATTGACCTG
CAGGAGCTCG GTAAATACGA ACAATACATT AAGTGGCCTT GGTATATATG GCTCGGCTTC
ATCGCGGGAC TGATCGCTAT CGTCATGGTT ACTATCATGC TGTGTTGCAT GACTTCATGT
TGCTCATGCC TTAAAGGATG CTGCTCCTGC GGGAGCTGTT GTAAGTTCGA CGAGGATGAT
TCAGAGCCGG TATTGAAAGG GGTAAAGCTC CACTATACTT GA

In accordance with one or more embodiments of the present application exemplary VLPs, expression plasmids, compositions, and methods are set out in the following items:
Item 1. A SARS-CoV-2 virus-like particle (VLP) comprising:
   a modified spike (S) glycoprotein of SARS-CoV-2, a matrix (M) protein of SARS-CoV-2, and an envelope (E) protein of SARS-CoV-2 wherein the modified S glycoprotein comprises an S1 domain and an S2 domain, and wherein the modified S glycoprotein includes at least one of the following:
      (i) linking the S1 and S2 domains via generation of disulfides bonds between the S1 and S2 domains,
      (ii) linking intra-polypeptide and inter-polypeptide S2 helices of the S2 domain, and
      (iii) substitution of one or more non-cysteine residues with a cysteine residue to generate one or more disulfide bonds
      wherein the modifications to the S glycoprotein (i) stabilizing a prefusion conformation of the S glycoprotein and/or (ii) prohibit a transition to a post-fusion structure.
Item 2. The SARS-CoV-2 VLP of item 1, wherein the linking of the S1 and S2 domains results from one or more of the following pairs of cysteine substitutions:
   (i) A653C at the S1 domain and A694C at the S2 domain;
   (ii) S659C at the S1 domain and S698C at the S2 domain; and
   (iii) C662C at the S1 domain and M697C at the S2 domain.
Item 3. The SARS-CoV-2 VLP of item 1 or 2, wherein the linking of intra-polypeptide and inter-polypeptide S2 helices of the S2 domain to one another results from one or more of the following pairs of cysteine substitutions:
   (i) Y707C and T883C at the S2 domain; and (ii) V705C and T883C at the S2 domain.
   Item 4. The SARS-CoV-2 VLP of any of items 1-3, wherein the substitution of one or more non-cysteine residues with a cysteine residue generates one or more disulfide bonds that prohibit the spike receptor binding domain (RBD) from a conformational change which includes one or more of the following substitutions:
(i) A570C at the S1 domain and V963C at the S2 domain;
(ii) D571C at the S1 domain and S967C at the S2 domain; and
(iii) K558C at the S1 domain and N282C at the S2 domain.
Item 5. The SARS-CoV-2 VLP of any of items 1-4, wherein the modifications to the S glycoprotein further include a P862C substitution at the S2 domain and A668C at the S1 domain, wherein these substitutions result in the locking of the S1 domain of one polypeptide chain to the S2 of another polypeptide chain, resulting in the stabilization of a prefusion conformation of the modified S glycoprotein.
Item 6. The SARS-CoV-2 VLP of any of items 1-5, further comprising an additional modification to the S glycoprotein, wherein the additional modification comprises replacing one or more domains of the SARS-CoV-2 S glycoprotein with analogous portions from one or more other coronaviruses producing a chimeric or mosaic S glycoprotein.
Item 7. The SARS-CoV-2 VLP of any of items 1-6, wherein the modified spike (S) glycoprotein is further coexpressed with a nucleocapsid (N) protein of SARS-CoV-2.
Item 8. The SARS-CoV-2 VLP of any of items 1-7, wherein the modifications to the S glycoprotein include at least one of the following pairs of cysteine substitutions:
   (i) A653C at the S1 domain and A694C at the S2 domain;
   (ii) S659C at the S1 domain and S698C at the S2 domain;
   (iii) C662C at the S1 domain and M697C at the S2 domain;
   (iv) V705C and T883C at the S2 domain;
   (v) A570C at the S1 domain and V963C at the S2 domain;
   (vi) D571C at the S1 domain and S967C at the S2 domain;
   (vii) Y707C and T883C at the S2 domain; and
   (vii) K558C at the S1 domain and N282C at the S2 domain.
Item 9. The SARS-CoV-2 VLP of any of items 1-9, wherein the SARS-CoV-2 VLP is suitable for the preparation of a SARS-CoV-2 vaccine.
Item 10. An expression plasmid comprising genes encoding coronavirus structural and surface proteins, wherein the expression plasmid is suitable for the assembly of the SARS-CoV-2 VLP of any of items 1-9, wherein the expression plasmid comprises optimized genes encoding a modified SARS-CoV-2 spike (S) glycoprotein, a SARS-CoV-2 matrix (M) protein, and a SARS-CoV-2 spike envelope (E) protein.
Item 11. The expression plasmid of item 10, wherein the expression plasmid further comprises optimized genes encoding a nucleocapsid (N) protein of SARS-CoV-2.
Item 12. A method for producing a SARS-CoV-2 VLP, the method comprising introducing into a host cell at least one expression plasmid of item 10 or 11 under conditions such that the host cell produces the SARS-CoV-2 VLP.
Item 13. The method of item 12, wherein the host cell is a eukaryotic cell.
Item 14. The method of item 13, wherein the eukaryotic cell is a mammalian cell.
Item 15. The method of item 14, wherein the eukaryotic cell is stably modified to continuously produce a VLP vaccine, such as a SARS-CoV-2 VLP vaccine.
Item 16. An immunogenic composition comprising at least one SARS-CoV-2 VLP of any of items 1-9.
Item 17. A method of generating an immune response to one or more coronaviruses in a subject, the method comprising administering an effective amount of the immunogenic composition of item 16.
Item 18. The method of item 17, wherein the composition is administered nasally, mucosally or parenterally.
Item 19. The method of item 17 or 18, wherein the subject is a human.
Item 20. The method of any of items 17-19, wherein the immune response vaccinates the subject against one or more coronaviruses.
Item 21. The method of any of items 17-20, wherein the immune response vaccinates the subject against SARS-CoV-2.
Item 22. The SARS-CoV-2 VLP of any preceding items further comprising at least one or more of the following mutations:
   (i) one or more amino acid residues at position 681-684 in an alpha variant;
   (ii) one or more amino acid residues at position 679-682 in a beta variant;
   (iii) one or more amino acid residues at position 682-685 in a delta variant;
   (iv) one or more amino acid residues at position 814-815;
   (v) one or more amino acid residues at position 983-984 in a beta variant;
   (vi) one or more amino acid residues at position 986-987 in a delta variant, and wherein for (i) to (iii), the one or more mutations are from RRAR to SGSA, and wherein for (iv) the one or more mutations are from KR to SG, and wherein for (v) to (vi) the one or more mutations are from KV to PP.
Item 23. An immunogenic composition comprising at least one SARS-CoV-2 VLP of any preceding items.
Item 24. A method of generating an immune response to one or more coronaviruses in a subject, the method comprising administering an effective amount of the immunogenic composition of any preceding items to the subject.
Item 25. An expression plasmid comprising genes encoding coronavirus structural and surface proteins, wherein the expression plasmid is suitable for the assembly of the SARS-CoV-2 VLP of any preceding items, wherein the expression plasmid comprises optimized genes encoding a modified SARS-CoV-2 spike (S) glycoprotein, a SARS-CoV-2 matrix (M) protein, and a SARS-CoV-2 spike envelope (E) protein.
Item 26. The expression plasmid of any preceding items, wherein the expression plasmid further comprises optimized genes encoding a nucleocapsid (N) protein of SARS-CoV-2.
Item 27. A method for producing a SARS-CoV-2 VLP, the method comprising introducing into a host cell at least one expression plasmid of any preceding items under conditions such that the host cell produces the SARS-CoV-2 VLP.

All publications, patents, and patent documents are incorporated by reference herein in their respective entireties, as though individually incorporated by reference. This statement of incorporation by reference is intended by applicants, pursuant to 37 C.F.R. § 1.57(b)(1), to relate to each and every individual publication, patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. No limitations inconsistent with this disclosure are to be understood therefrom.

The invention has been described with reference to various specific and preferred embodiments and techniques. However, it should be understood that many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims. Such equivalents are intended to be encompassed by the following claims.

What is claimed:

1. A SARS-CoV-2 virus-like particle (VLP) comprising:
a modified spike (S) glycoprotein of SARS-CoV-2, a matrix (M) protein of SARS-CoV-2, and an envelope (E) protein of SARS-CoV-2 wherein the modified S glycoprotein comprises an S1 domain and an S2 domain, and wherein the modified S glycoprotein includes at least one of the following modifications:
(i) linking the S1 and S2 domains via generation of disulfides bonds between the S1 and S2 domains,
(ii) linking intra-polypeptide and inter-polypeptide S2 helices of the S2 domain, and
(iii) substitution of one or more non-cysteine residues with a cysteine residue to generate one or more disulfide bonds
wherein the modifications to the S glycoprotein (i) stabilize a prefusion conformation of the S glycoprotein and/or (ii) prohibit a transition to a post-fusion structure.

2. The SARS-CoV-2 VLP of claim 1, wherein the linking of the S1 and S2 domains results from one or more of the following pairs of cysteine substitutions:
(i) A653C at the S1 domain and A694C at the S2 domain;
(ii) S659C at the S1 domain and S698C at the S2 domain; and
(iii) C662C at the S1 domain and M697C at the S2 domain.

3. The SARS-CoV-2 VLP of claim 1, wherein the linking of intra-polypeptide and inter-polypeptide S2 helices of the S2 domain to one another results from one or more of the following pairs of cysteine substitutions:
(i) Y707C and T883C at the S2 domain; and
(ii) V705C and T883C at the S2 domain.

4. The SARS-CoV-2 VLP of claim 1, wherein the substitution of one or more non-cysteine residues with a cysteine residue generates one or more disulfide bonds that prohibit the spike receptor binding domain (RBD) from a conformational change which includes one or more of the following substitutions:
(i) A570C at the S1 domain and V963C at the S2 domain;
(ii) D571C at the S1 domain and S967C at the S2 domain; and
(iii) K558C at the S1 domain and N282C at the S2 domain.

5. The SARS-CoV-2 VLP of claim 1, wherein the modifications to the S glycoprotein further include a P862C substitution at the S2 domain and A668C at the S1 domain, wherein these substitutions result in the locking of the S1 domain of one polypeptide chain to the S2 of another polypeptide chain, resulting in the stabilization of a prefusion conformation of the modified S glycoprotein.

6. The SARS-CoV-2 VLP of claim 1, further comprising an additional modification to the S glycoprotein, wherein the additional modification comprises replacing one or more domains of the SARS-CoV-2 S glycoprotein with analogous portions from one or more other coronaviruses producing a chimeric or mosaic S glycoprotein.

7. The SARS-CoV-2 VLP of claim 1, wherein the modified spike (S) glycoprotein is further coexpressed with a nucleocapsid (N) protein of SARS-CoV-2.

8. The SARS-CoV-2 VLP of claim 1, wherein the modifications to the S glycoprotein include at least one of the following pairs of cysteine substitutions:
(i) A653C at the S1 domain and A694C at the S2 domain;
(ii) S659C at the S1 domain and S698C at the S2 domain;
(iii) C662C at the S1 domain and M697C at the S2 domain;
(iv) V705C and T883C at the S2 domain;
(v) A570C at the S1 domain and V963C at the S2 domain;
(vi) D571C at the S1 domain and S967C at the S2 domain;
(vii) Y707C and T883C at the S2 domain; and
(vii) K558C at the S1 domain and N282C at the S2 domain.

9. The SARS-CoV-2 VLP of claim 1, wherein the SARS-CoV-2 VLP is suitable for the preparation of a SARS-CoV-2 vaccine.

10. An expression plasmid comprising genes encoding coronavirus structural and surface proteins, wherein the expression plasmid is suitable for the assembly of the SARS-CoV-2 VLP of claim 1, wherein the expression plasmid comprises optimized genes encoding a modified SARS-CoV-2 spike (S) glycoprotein, a SARS-CoV-2 matrix (M) protein, and a SARS-CoV-2 spike envelope (E) protein.

11. The expression plasmid of claim 10, wherein the expression plasmid further comprises optimized genes encoding a nucleocapsid (N) protein of SARS-CoV-2.

12. A method for producing a SARS-CoV-2 VLP, the method comprising introducing into a host cell at least one expression plasmid of claim 10 under conditions such that the host cell produces the SARS-CoV-2 VLP.

13. The method of claim 12, wherein the host cell is a eukaryotic cell.

14. The method of claim 13, wherein the eukaryotic cell is a mammalian cell.

15. The method of claim 14, wherein the eukaryotic cell is stably modified to continuously produce a VLP vaccine.

16. An immunogenic composition comprising at least one SARS-CoV-2 VLP of claim 1.

17. A method of generating an immune response to one or more coronaviruses in a subject, the method comprising administering an effective amount of the immunogenic composition of claim 16 to the subject.

18. The method of claim 17, wherein the composition is administered nasally, mucosally or parenterally.

19. The method of claim 17, wherein the subject is a human.

20. The method of claim 17, wherein the immune response vaccinates the subject against one or more coronaviruses.

21. The method of claim 20, wherein the immune response vaccinates the subject against SARS-CoV-2.

22. The SARS-CoV-2 VLP of claim 1 further comprising at least one or more of the following mutations:
(i) one or more amino acid residues at position 681-684 in an alpha variant;
(ii) one or more amino acid residues at position 679-682 in a beta variant;
(iii) one or more amino acid residues at position 682-685 in a delta variant;
(iv) one or more amino acid residues at position 814-815;
(v) one or more amino acid residues at position 983-984 in a beta variant;
(vi) one or more amino acid residues at position 986-987 in a delta variant,
and wherein for (i) to (iii), the one or more mutations are from RRAR to SGSA, and wherein for (iv) the one or more mutations are from KR to SG, and wherein for (v) to (vi) the one or more mutations are from KV to PP.

23. An immunogenic composition comprising at least one SARS-CoV-2 VLP of claim 22.

24. A method of generating an immune response to one or more coronaviruses in a subject, the method comprising administering an effective amount of the immunogenic composition of claim 23 to the subject.

25. An expression plasmid comprising genes encoding coronavirus structural and surface proteins, wherein the expression plasmid is suitable for the assembly of the SARS-CoV-2 VLP of claim 22, wherein the expression plasmid comprises optimized genes encoding a modified SARS-CoV-2 spike (S) glycoprotein, a SARS-CoV-2 matrix (M) protein, and a SARS-CoV-2 spike envelope (E) protein.

26. The expression plasmid of claim 25, wherein the expression plasmid further comprises optimized genes encoding a nucleocapsid (N) protein of SARS-CoV-2.

27. A method for producing a SARS-CoV-2 VLP, the method comprising introducing into a host cell at least one expression plasmid of claim 25 under conditions such that the host cell produces the SARS-CoV-2 VLP.
